(19) United States
(12) Patent Application Publication
(10) Pub. No.: US 2009/0318446 A1
(43) Pub. Date: Dec. 24, 2009

(54) 4-(1H-Indol-3-yl)-Pyrimidin-2-Ylamine Derivatives and Their Use in Therapy

(75) Inventors: Peter Martin Fischer, Nottingham (GB); Shudong Wang, Nottingham (GB); Christopher Meades, Dundee (GB); Matin J.I. Andrews, Perk (BE); Darren Gibson, Dundee (GB); Kenneth Duncan, Cambusbarron (GB)

Correspondence Address: LAHIVE & COCKFIELD, LLP;FLOOR 30, SUITE 3000, ONE POST OFFICE SQUARE, BOSTON, MA, 02109, US

(73) Assignee: Cyclacel Limited, Dundee (GB)

(21) Appl. No.: 11/794,449

(22) PCT Filed: Jan. 11, 2006
(86) PCT No.: PCT/GB2006/000087

§ 371 Date: Sep. 9, 2009

(30) Foreign Application Priority Data
Jan. 11, 2005 (GB) 0500492.4

(51) Int. Cl. A61K 31/506; C07D 403/04; C07D 401/14; C07D 413/14; A61K 31/5377; A61P 35/00; A61P 11/00; G01N 33/53

(52) U.S. Cl. 514/235.2; 544/331; 544/122; 544/295; 514/275; 514/252.19; 435/7.93

(57) ABSTRACT

The present invention relates to compounds of formula (I), or pharmaceutically acceptable salts thereof. The present invention seeks to provide further substituted heteroaryl-substituted pyrimidine derivatives. More specifically, the invention relates to compounds that have broad therapeutic applications in the treatment of a number of different diseases and/or that are capable of inhibiting one or more protein kinases.

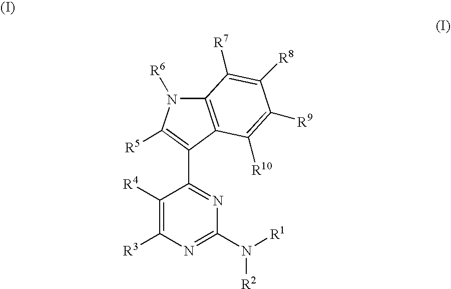

The present invention relates to substituted pyrimidine derivatives. In particular, the invention relates to 4-(1H-indol-3-yl)-pyrimidin-2-ylamines and their use in therapy. More specifically, but not exclusively, the invention relates to compounds that are capable of inhibiting one or more protein kinases.

### BACKGROUND TO THE INVENTION

In eukaryotes, all biological functions, including DNA replication, cell cycle progression, energy metabolism, and cell growth and differentiation, are regulated through the reversible phosphorylation of proteins. The phosphorylation state of a protein determines not only its function, subcellular distribution, and stability, but also what other proteins or cellular components it associates with. The balance of specific phosphorylation in the proteome as a whole, as well as of individual members in a biochemical pathway, is thus used by organisms as a strategy to maintain homeostasis in response to an ever-changing environment [71]. The enzymes that carry out these phosphorylation and dephosphorylation steps are protein kinases and phosphatases, respectively. Many kinases have gained importance as drug discovery targets in a variety of therapeutic areas [72].

The eukaryotic protein kinase family is one of the largest in the human genome, comprising some 500 genes [1,2]. The majority of kinases contain a 250-300 amino acid residue catalytic domain with a conserved core structure. This domain comprises a binding pocket for ATP (less frequently GTP), whose terminal phosphate group the kinase transfers covalently to its macromolecular substrates. The phosphate donor is always bound as a complex with a divalent ion (usually Mg²⁺ or Mn²⁺). Another important function of the catalytic domain is the binding and orientation for phosphotransfer of the macromolecular substrate. The catalytic domains present in most kinases are more or less homologous.

A wide variety of molecules capable of inhibiting protein kinase function through antagonising ATP binding are known in the art [3-7]. By way of example, the applicant has previously disclosed 2-anilino-4-heteroaryl-pyrimidine compounds with kinase inhibitory properties, particularly against cyclin-dependent kinases (CDKs) [8-12]. CDKs are serine/threonine protein kinases that associate with various cyclin subunits. These complexes are important for the regulation of eukaryotic cell cycle progression, but also for the regulation of transcription [13,14].

The present invention seeks to provide further substituted heteroaryl-substituted pyrimidine derivatives. More specifically, the invention relates to compounds that have broad therapeutic applications in the treatment of a number of different diseases and/or that are capable of inhibiting one or more protein kinases.

### STATEMENT OF INVENTION

A first aspect of the invention relates to 4-(1H-indol-3-yl)-pyrimidin-2-ylamines. More specifically, the invention relates to compounds of formula I, or pharmaceutically acceptable salts thereof,

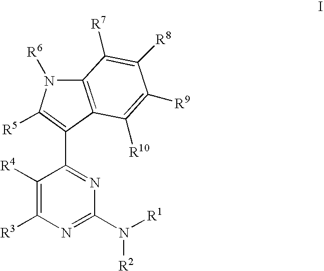

wherein R³, R⁴, R⁵, R⁶, R⁷, R⁸, R⁹, and R¹⁰ are each independently H, R¹¹ or R¹²;
R¹ and R² are each independently H, R¹¹ or R¹²; or R¹ and R² are linked to form a cyclic group together with the nitrogen to which they are attached, and wherein said cyclic group is optionally substituted with one or more R¹¹ or R¹² groups;
each R¹¹ is independently a hydrocarbyl group optionally substituted by one or more R¹² substituents;
each R¹² is independently selected from OR¹³, COR¹³, COOR¹³, CN, CONR¹³R¹⁴, NR¹³R¹⁴, SR¹³, SOR¹³, SO₂R¹³, SO₂OR¹³, SO₂NR¹³R¹⁴, R¹³, halogen, CF₃, NO₂ and an alicyclic group itself optionally substituted by one or more R¹² or R¹³ groups; and
each R¹³ and each R¹⁴ are independently H or (CH₂)ₙR¹⁵ where n is 0, 1, 2, or 3; and
each R¹⁵ is independently selected from alkyl, cycloalkyl, aryl, heteroaryl, aryl and an alicyclic group;
with the proviso that the compound is other than:
[4-(1H-indol-3-yl)-pyrimidin-2-yl]-[3-(1,1,2,2-tetrafluoroethoxyphenyl)]-amine;
3-[6-(4-bromophenyl)-2-(1-piperazinyl)-4-pyrimidinyl]-1H-indole;
3-[6-(4-bromophenyl)-2-(1-pyrrolidinyl)-4-pyrimidinyl]-1H-indole; or
3-[6-(4-bromophenyl)-2-(4-morpholinyl)-4-pyrimidinyl]-1H-indole.

Several 4-(1H-indol-3-yl)-pyrimidin-2-ylamine compounds are known in the art [73]. However, to date, the only such compound reported to inhibit kinase activity is [4-(1H-indol-3-yl)-pyrimidin-2-yl]-[3-(1,1,2,2-tetrafluoro-ethoxy)-phenyl]-amine [74], which was shown to inhibit PKC-α, PKC-δ and EGF-R.

The present invention provides compounds that are capable of inhibiting various other protein kinases, including aurora kinase [75], FMS-like tyrosine kinase 3 (FLT3) [76], cyclin-dependent kinases (CDKS) [77], and glycogen synthase kinase 3 (GSK3) [78].

A second aspect of the invention relates to a pharmaceutical composition comprising a compound of formula I as defined above, or a pharmaceutically acceptable salt thereof, admixed with a pharmaceutically acceptable diluent, excipient or carrier.

A third aspect of the invention relates to the use of a compound of formula Ia, or a pharmaceutically acceptable salt thereof,

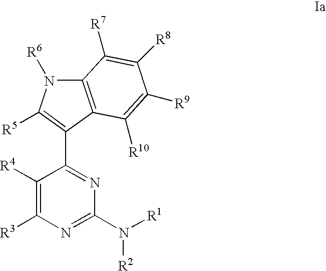

wherein R³, R⁴, R⁵, R⁶, R⁷, R⁸, R⁹, and R¹⁰ are each independently H, R¹¹ or R¹²;
R¹ and R² are each independently H, R¹¹ or R¹²; or R¹ and R² are linked to form a cyclic group together with the nitrogen to which they are attached, and wherein said cyclic group is optionally substituted with one or more R¹¹ or R¹² groups;
each R¹¹ is independently a hydrocarbyl group optionally substituted by one or more R¹² substituents;
each R¹² is independently selected from OR¹³, COR¹³, COOR¹³, CN, CONR¹³R¹⁴, NR¹³R¹⁴, SR¹³, SOR¹³, SO₂R¹³, SO₂OR¹³, SO₂NR¹³R¹⁴, R¹³, halogen, CF₃, NO₂ and an alicyclic group itself optionally substituted by one or more R¹² or R¹³ groups; and
each R¹³ and each R¹⁴ are independently H or (CH₂)ₙR¹⁵, where n is 0, 1, 2, or 3; and
each R¹⁵ is independently selected from alkyl, cycloalkyl, aryl, heteroaryl, aryl and an alicyclic group;
with the proviso that the compound is other than [4-(1H-indol-3-yl)-pyrimidin-2-yl]-[3-(1,1,2,2-tetrafluoroethoxyphenyl)]-amine;
in the preparation of a medicament for treating a proliferative disorder.

Further aspects of the invention relate to the use of a compound of formula Ib, or a pharmaceutically acceptable salt thereof,

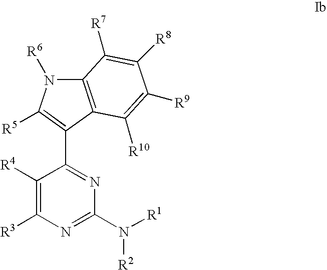

wherein R³, R⁴, R⁵, R⁶, R⁷, R⁸, R⁹, and R¹⁰ are each independently H, R¹¹ or R¹²;
R¹ and R² are each independently H, R¹¹ or R¹²; or R¹ and R² are linked to form a cyclic group together with the nitrogen to which they are attached, and wherein said cyclic group is optionally substituted with one or more R¹¹ or R¹² groups;
each R¹¹ is independently a hydrocarbyl group optionally substituted by one or more R¹² substituents;
each R¹² is independently selected from OR¹³, COR¹³, COOR¹³, CN, CONR¹³R¹⁴, NR¹³R¹⁴, SR¹³, SOR¹³, SO₂R¹³, SO₂OR¹³, SO₂NR¹³R¹⁴, R¹³, halogen, CF₃, NO₂ and an alicyclic group itself optionally substituted by one or more R¹² or R¹³ groups; and
each R¹³ and each R¹⁴ are independently H or (CH₂)ₙR¹⁵, where n is 0, 1, 2, or 3; and
each R¹⁵ is independently selected from alkyl, cycloalkyl, aryl, heteroaryl, aryl and an alicyclic group;
in the preparation of a medicament for treating one or more of the following:
a viral disorder;
a CNS disorder;
a stroke;
a microbial infection;
a fungal disorder;
a parasitic disorder;
an inflammatory disorder;
a cardiovascular disorder;
alopecia; and
diabetes.

Another aspect of the invention relates to the use of a compound of formula Ib as defined above, or a pharmaceutically acceptable salt thereof in an assay for identifying further candidate compounds capable of inhibiting one or more of a cyclin dependent kinase, GSK, aurora kinase, FLT3 and a PLK enzyme.

Another aspect of the invention relates to compounds of formula I as defined above, or pharmaceutically acceptable salts thereof, for use in medicine.

A further aspect of the invention relates to a process for preparing compounds according to the invention.

### DETAILED DESCRIPTION

For the avoidance of doubt, the preferred embodiments described hereinafter refer to all aspects of the present invention.

As used herein, the term “hydrocarbyl” refers to a group comprising at least C and H. If the hydrocarbyl group comprises more than one C then those carbons need not necessarily be linked to each other. For example, at least two of the carbons may be linked via a suitable element or group. Thus, the hydrocarbyl group may contain heteroatoms. Suitable heteroatoms will be apparent to those skilled in the art and include, for instance, sulphur, nitrogen, oxygen, phosphorus and silicon. Where the hydrocarbyl group contains one or more heteroatoms, the group may be linked via a carbon atom or via a heteroatom to another group, i.e. the linker atom may be a carbon or a heteroatom. Preferably, the hydrocarbyl group is an aryl, heteroaryl, alkyl, cycloalkyl, aralkyl, alicyclic, heteroalicyclic or alkenyl group. More preferably, the hydrocarbyl group is an aryl, heteroaryl, alkyl, cycloalkyl, aralkyl or alkenyl group. The hydrocarbyl group may be optionally substituted by one or more R¹² groups.

As used herein, the term “alkyl” includes both saturated straight chain and branched alkyl groups which may be substituted (mono- or poly-) or unsubstituted. Preferably, the alkyl group is a C₁₋₂₀ alkyl group, more preferably a C₁₋₁₅, more preferably still a C₁₋₁₂ alkyl group, more preferably still, a C₁₋₆ alkyl group, more preferably a C₁₋₃ alkyl group. Particularly preferred alkyl groups include, for example, methyl, ethyl, propyl, isopropyl, butyl, isobutyl, tert-butyl, pentyl and hexyl. Suitable substituents include, for example, one or more R¹² groups. Preferably, the alkyl group is unsubstituted.

As used herein, the term “cycloalkyl” refers to a cyclic alkyl group which may be substituted (mono- or poly-) or unsubstituted. Preferably, the cycloalkyl group is a C₃₋₁₂ cycloalkyl group. Suitable substituents include, for example, one or more R¹² groups.

As used herein, the term “alkenyl” refers to a group containing one or more carbon-carbon double bonds, which may be branched or unbranched, substituted (mono- or poly-); or unsubstituted. Preferably the alkenyl group is a C₂₋₂₀ alkenyl group, more preferably a C₂₋₁₅ alkenyl group, more preferably still a C₂₋₁₂ alkenyl group, or preferably a C₂₋₆ alkenyl group, more preferably a C₂₋₃ alkenyl group. Suitable substituents include, for example, one or more R¹² groups as defined above.

As used herein, the term “aryl” refers to a C₆₋₁₂ aromatic group which may be substituted (mono- or poly-) or unsubstituted. Typical examples include phenyl and naphthyl etc. Suitable substituents include, for example, one or more R¹² groups.

As used herein, the term “heteroaryl” refers to a C₂₋₁₂ aromatic, substituted (mono- or poly-) or unsubstituted group, which comprises one or more heteroatoms. Preferably, the heteroaryl group is a C₄₋₁₂ aromatic group comprising one or more heteroatoms selected from N, O and S. Suitable heteroaryl groups include pyrrole, pyrazole, pyrimidine, pyrazine, pyridine, quinoline, thiophene, 1,2,3-triazole, 1,2,4-triazole, thiazole, oxazole, iso-thiazole, iso-oxazole, imidazole, furan and the like. Again, suitable substituents include, for example, one or more R¹² groups.

As used herein, the term “alicyclic” refers to a cyclic aliphatic group which optionally contains one or more heteroatoms and which may be substituted (mono- or poly-) or unsubstituted. Preferably, the alicyclic group contains one or more heteroatoms and is thus a heteroalicylic group. Preferred heteroalicyclic groups include piperidinyl, pyrrolidinyl, piperazinyl, thiomorpholinyl and morpholinyl. More preferably, the heteroalicyclic group is selected from N-piperidinyl, N-pyrrolidinyl, N-piperazinyl, N-thiomorpholinyl and N-morpholinyl. Again, suitable substituents include, for example, one or more R¹² groups.

As used herein, the term “aralkyl” includes, but is not limited to, a group having both aryl and alkyl functionalities. By way of example, the term includes groups in which one of the hydrogen atoms of the alkyl group is replaced by an aryl group, e.g. a phenyl group optionally having one or more substituents such as halo, alkyl, alkoxy, hydroxy, and the like. Typical aralkyl groups include benzyl, phenethyl and the like.

As used herein, the term “aryl-alicyclic” includes, but is not limited to, a group having both aryl and alicyclic functionalities. By way of example the term includes groups which contain an aryl functionality (for example, a phenyl group) fused to an alicylic group. The alicylic group may contain one or more heteroatoms, i.e. it may be a heteroalicylic group.

One preferred embodiment of the invention relates to compounds of formula I, or pharmaceutically acceptable salts thereof,

wherein R³, R⁴, R⁵, R⁶, R⁷, R⁸, R⁹, and R¹⁰ are each independently H, R¹¹ or R¹²;
R¹ and R² are each independently H, R¹¹ or R¹²; or R¹ and R² are linked to form a cyclic group together with the nitrogen to which they are attached, and wherein said cyclic group is optionally substituted with one or more R¹¹ or R¹² groups;
each R¹¹ is independently a hydrocarbyl group optionally substituted by one or more R¹² substituents;
each R¹² is independently selected from OR¹³, COR¹³, COOR¹³, CN, CONR¹³R¹⁴, NR¹³R¹⁴, SR¹³, SOR¹³, SO₂R¹³, SO₂OR¹³, SO₂NR¹³R¹⁴, an alicyclic group, halogen, CF₃, and NO₂; and
R¹³ and R¹⁴ are each independently H or (CH₂)ₙR¹⁵, where n is 0, 1, 2, or 3; and
each R¹⁵ is independently selected from alkyl, cycloalkyl, aryl, heteroaryl, aryl and an alicyclic group;
with the proviso that the compound is other than:
[4-(1H-indol-3-yl)-pyrimidin-2-yl]-[3-(1,1,2,2-tetrafluoroethoxyphenyl)]-amine;
3-[6-(4-bromophenyl)-2-(1-piperazinyl)-4-pyrimidinyl]-1H-indole;
3-[6-(4-bromophenyl)-2-(1-pyrrolidinyl)-4-pyrimidinyl]-1H-indole; or
3-[6-(4-bromophenyl)-2-(4-morpholinyl)-4-pyrimidinyl]-1H-indole.

In one preferred embodiment of the invention, R¹ and R² are each independently H, R¹¹ or R¹²; or R¹ and R² are linked to form a cyclic group together with the nitrogen to which they are attached, wherein said cyclic group contains from two to nine carbon atoms and one or two heteroatoms selected from N, O, and S, and wherein said cyclic group is optionally substituted with one or two substituents selected from R¹¹ and R¹².

In one preferred embodiment of the invention, R¹ and R² are each independently H, R¹¹ or R¹²

In a more preferred embodiment of the invention, R¹ and R² are each independently H or R¹¹.

In one particularly preferred embodiment of the invention, one of R¹ and R² is H and the other is R¹¹.

In another particularly preferred embodiment, R¹ and R² are both H.

Preferably, R¹¹ is a hydrocarbyl group containing from 1 to 24 carbon atoms, optionally containing up to six heteroatoms selected from N, O, and S.

More preferably, the hydrocarbyl group is optionally substituted by up to six R¹² substituents.

In one preferred embodiment, R¹¹ is an aryl group, a heteroaryl group, an aryl-alicyclic group or an alicyclic group, each of which may be optionally substituted by one or more R¹² substituents.

In one preferred embodiment, R¹¹ is selected from phenyl, pyridinyl and

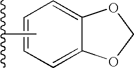

each of which may be optionally substituted by one or more R¹² substituents.

In one preferred embodiment, R¹¹ is an aryl, heteroaryl or alicyclic group, each of which may be optionally substituted by one or more R¹² substituents.

In an even more preferred embodiment, R¹¹ is a phenyl or pyridinyl group, each of which may be optionally substituted by one or more R¹² substituents.

In one preferred embodiment R³, R⁴, R⁵, R⁶, R⁷, R⁸, R⁹, and R¹⁰ are each independently H or R¹².

Preferably, R³ is H and R⁴ is H or R¹².

Preferably, R³ and R⁴ are both H.

Preferably, R⁹ and R¹⁰ are both H.

In another preferred embodiment, R⁵ is H or alkyl, more preferably, H or Me.

In another preferred embodiment, R⁶ is H, alkyl, CO-alkyl or CO-cycloalkyl, and is more preferably, H, Me, COMe or CO-cyclopropyl. More preferably still, R⁶ is H.

In another preferred embodiment, R⁷ is H, alkyl, alkoxy or halo, more preferably, H, Me, OMe or chloro.

In another preferred embodiment, R⁸ is H, alkoxy or halo, more preferably, H, OMe or F.

In another preferred, embodiment R⁵, R⁶, R⁷, R⁸, R⁹, and R¹⁰ are all H.

In one preferred embodiment, each R¹⁵ is independently selected from methyl, ethyl, isopropyl, n-butyl, isobutyl, t-butyl, cyclopropyl, cyclobutyl, cyclopentyl, cyclohexyl, phenyl, pyridinyl, pyrrolidinyl, pyrrolyl, morpholinyl, piperazinyl, piperidinyl, thiazolyl, tetrazolyl and thiazolyl. More preferably, each R¹⁵ is alkyl or aryl.

In one highly preferred embodiment, R¹⁵ is Me or phenyl, more preferably, Me.

In one preferred embodiment, the alicyclic group contains one or more heteroatoms.

In one preferred embodiment, R¹² is an alicyclic group optionally substituted by one or more R¹³ or COR¹³ groups.

In a more preferred embodiment, R¹² is a morpholinyl, piperazinyl, thiomorpholinyl or piperidinyl group optionally substituted by one or more R¹³ or COR¹³ groups.

In an even more preferred embodiment, R¹² is a morpholinyl, piperazinyl, thiomorpholinyl or piperidinyl group optionally substituted by one or more alkyl, aralkyl or CO-alkyl groups.

More preferably, R¹² is a morpholinyl, piperazinyl, thiomorpholinyl or piperidinyl group optionally substituted by one or more methyl, benzyl or COMe groups.

More preferably still, R¹² is selected from the following:

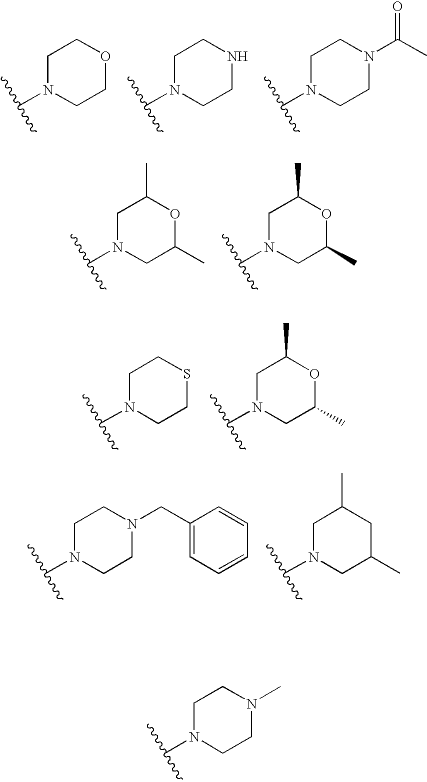

In one preferred embodiment, each R¹² is independently selected from OR¹³, COR¹³, COOR¹³, CN, CONR¹³R¹⁴, NR¹³R¹⁴, SR¹³, SOR¹³, SO₂R¹³, SO₂OR¹³, SO₂NR¹³R¹⁴, a heteroalicyclic group, halogen, CF₃, and NO₂.

In a more preferred embodiment, each R¹² is independently selected from OH, OMe, COMe, CHO, CO₂Me, COOH, CN, CONH₂, NHMe, NH₂, NMe₂, SH, SMe, SOMe, SO₂Me, SO₂NHMe, SO₂NH₂, Cl, Br, F, I, CF₃, NO₂, N-morpholinyl, N-pyrrolidinyl and N-piperazinyl, N-thiomorpholinyl, 2,6-dimethylmorpholin-4-yl, 4-benzylpiperazin-1-yl, 3,5-dimethylpiperidin-1-yl and 4-acetylpiperazin-1-yl.

In an even more preferred embodiment, each R¹² is independently selected from OH, OMe, COMe, CHO, CO₂Me, COOH, CN, CONH₂, NHMe, NH₂, NMe₂, SH, SMe, SOMe, SO₂Me, SO₂NHMe, SO₂NH₂, Cl, Br, F, I, CF₃, NO₂, N-morpholinyl, N-pyrrolidinyl and N-piperazinyl.

In one highly preferred embodiment, R¹² is selected from

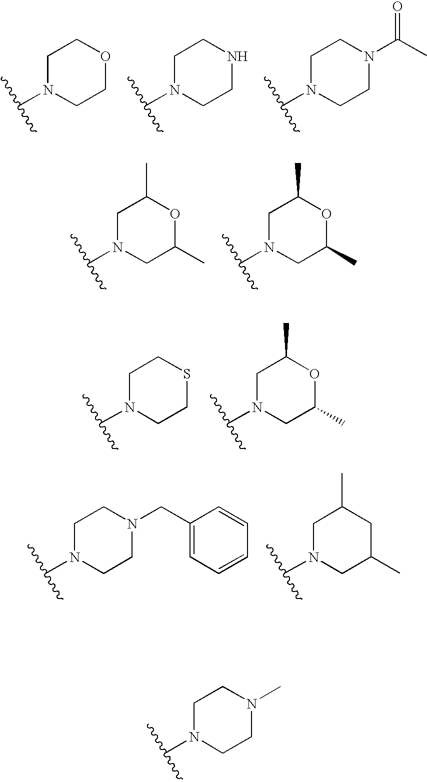

### NO₂, F, OMe, NMe₂ and Me.

In one preferred embodiment, R¹³ is (CH₂)ₙR¹⁵ where n is 0 or 1. More preferably, n is 0.

One particularly preferred embodiment of the invention relates to compounds of formula Ic, or pharmaceutically acceptable salts thereof,

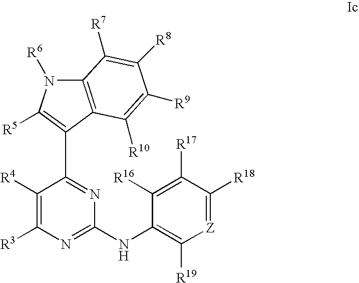

wherein
R³⁻¹⁰ are as defined above;

### Z is N or CR²⁰; and

R¹⁶⁻²⁰ are each independently H, R¹¹ or R¹².

In one preferred embodiment, Z is N.

In another preferred embodiment, Z is CR²⁰.

In one preferred embodiment, R¹⁶⁻²⁰ are each independently selected from H and R¹² as defined above.

In one particularly preferred embodiment, R¹⁶⁻²⁰ are each independently selected from H, NO₂, NR¹³R¹⁴, halogen, alkoxy and an optionally substituted heteroalicyclic group.

In a more preferred embodiment, R¹⁶⁻²⁰ are each independently selected from H, NO₂, halogen, alkoxy and a heteroalicyclic group.

In one preferred embodiment, R¹⁶⁻²⁰ are each independently selected from H, NO₂, F, OMe, N-morpholinyl, NH₂, N-pyrrolidinyl, N-piperazinyl, N-thiomorpholinyl, 2,6-dimethylmorpholin-4-yl, 4-benzylpiperazin-1-yl, 3,5-dimethyl-piperidin-1-yl and 4-acetylpiperazin-1-yl.

More preferably, R¹⁶⁻²⁰ are each independently selected from H, NO₂, F, OMe and N-morpholinyl.

In one especially preferred embodiment, the compound of the invention is selected from the following:
4-(1H-indol-3-yl)-N-(3-nitrophenyl)pyrimidin-2-amine;
N-(4-fluorophenyl)-4-(1H-indol-3-yl)pyrimidin-2-amine;
4-(1H-indol-3-yl)-N-(6-methoxypyridin-3-yl)pyrimidin-2-amine;
4-(1H-indol-3-yl)-N-(4-morpholinylphenyl)pyrimidin-2-amine;
4-(1H-indol-3-yl)-N-(4-acetylpiperazin-1-ylphenyl)pyrimidin-2-amine;
4-(1H-indol-3-yl)-N-(4-piperazin-1-ylphenyl)pyrimidin-2-amine;
4-(1H-indol-3-yl)-N-(4-benzylpiperazin-1-ylphenyl)pyrimidin-2-amine;
4-(1H-indol-3-yl)-N-(2,6-dimethylmorpholin-4-ylphenyl)pyrimidin-2-amine;
N′-[4-(1H-indol-3-yl)pyrimidin-2-yl]-N,N-dimethylbenzene-1,4-diamine;
4-(1H-indol-3-yl)-N-(2-methyl-4-morpholin-4-ylphenyl)pyrimidin-2-amine;
4-(1H-indol-3-yl)-N-(3,4,5-trimethoxyphenyl)pyrimidin-2-amine;
4-(1H-indol-3-yl)-N-(3-methoxyl-4-morpholin-4-ylphenyl)pyrimidin-2-amine;
N-(3,5-dimethoxyphenyl)-4-(1H-indol-3-yl)pyrimidin-2-amine;
4-(1-methyl-1H-indol-3-yl)-N-(4-morpholin-4-ylphenyl)pyrimidin-2-amine;
4-(1-methyl-1H-indol-3-yl)-N-(4-acetylpiperazine-1-ylphenyl)pyrimidin-2-amine;
N-1,3-benzodioxol-5-yl-4-(1H-indol-3-yl)pyrimidin-2-amine;
4-[1-(cyclopropylcarbonyl)-1H-indol-3-yl]-N-(4-morpholin-4ylphenyl)pyrimidin-2-amine;
4-(1-acetyl-1H-indol-3-yl)-N-(4-morpholin-4ylphenyl)pyrimidin-2-amine;
4-(1H-indol-3-yl)-N-(4-methylpiperazin-1-ylphenyl)pyrimidin-2-amine;
4-(7-methoxy-1H-indol-3-yl)-N-(4-acetylpiperazin-1-ylphenyl)pyrimidin-2-amine;
4-(2-methyl-1H-indol-3-yl)-N-(4-acetylpiperazin-1-ylphenyl)]pyrimidin-2-amine;
4-(7-methyl-1H-indol-3-yl)-N-(4-acetylpiperazin-1-ylphenyl)]pyrimidin-2-amine;
4-(6-methoxy-1H-indol-3-yl)-N-(4-acetylpiperazin-1-ylphenyl)]pyrimidin-2-amine;
4-(7-chloro-1H-indol-3-yl)-N-(4-acetylpiperazin-1-ylphenyl)]pyrimidin-2-amine;
4-(6-fluoro-1H-indol-3-yl)-N-(4-acetylpiperazin-1-ylphenyl)]pyrimidin-2-amine;
4-(1H-indol-3-yl)-N-[(4-acetylpiperazin-1-yl)-3-methylphenyl]pyrimidin-2-amine;
4-(1H-indol-3-yl)-N-(3-methyl-4-thiomorpholin-4-ylphenyl)pyrimidin-2-amine;
4-(1H-indol-3-yl)-N-[(2R,6S)-2,6-dimethylmorpholin-4-ylphenyl]pyrimidin-2-amine;
4-(1H-indol-3-yl)-N-[(2S,6S)-2,6-dimethylmorpholin-4-ylphenyl]pyrimidin-2-amine;
4-(1H-indol-3-yl)-N-(3,5-dimethylpiperidin-1-ylphenyl)pyrimidin-2-amine; and
4-(1H-indol-3-yl)pyrimidin-2-amine.

In one particularly preferred embodiment, the compound is selected from the following:
4-(1H-Indol-3-yl)-pyrimidin-2-ylamine;
[4-(1H-Indol-3-yl)-pyrimidin-2-yl]-(3-nitro-phenyl)-amine;
(4-Fluoro-phenyl)-[4-(1H-indol-3-yl)-pyrimidin-2-yl]-amine;
[4-(1H-Indol-3-yl)-pyrimidin-2-yl]-(6-methoxy-pyridin-3-yl)-amine; and
[4-(1H-Indol-3-yl)-pyrimidin-2-yl]-(4-morpholin-4-yl-phenyl)-amine.

In one preferred embodiment, the compound of the invention is capable of inhibiting one or more protein kinases selected from CDK1/cyclin B, CDK2/cyclin A, CDK2/cyclin E, CDK4/cyclin D1, CDK7/cyclin H, CDK9/cyclin T1, GSK3β, aurora kinase, FLT3 and PLK1, as measured by the appropriate assay.

In one particularly preferred embodiment, the compound of the invention exhibits an IC₅₀ value for kinase inhibition of less than about 10 μM, more preferably less than about 5 μM, more preferably less than about 1 μM, more preferably still less than about 0.5 μM, more, preferably less than about 0.1 μM, even more preferably, less than about 0.01 μM. Compounds falling within each of these preferred embodiments can be identified from Tables 2 and 3, which show the IC₅₀ values for selected compounds of the invention. Details of the various kinase assays are disclosed in the accompanying Examples section. Compounds (12) and (13) are especially preferred in this regard.

In one preferred embodiment the compound of the invention is capable of exhibiting an antiproliferative effect in human cell lines, as measured by a standard 72 h MTT cytotoxicity assay. Preferably, the compound of the invention exhibits an IC₅₀ value of less than 10 μM, more preferably less than 5 μM, even more preferably less than 1 μM as measured by said MTT assay. More preferably still, the compound exhibits an IC₅₀ value of less than 0.5 less μM, more preferably still less than 0.2 μM or 0.1 μM. Compounds falling within each of these preferred embodiments can be identified from Table 4, which show the IC₅₀ values for selected compounds of the invention. Details of the standard 72 h MTT cytotoxicity assay are set forth in the accompanying Examples section. Compound (12) is especially preferred in this regard.

### Therapeutic Use

The compounds of the present invention have been found to possess anti-proliferative activity and are therefore believed to be of use in the treatment of proliferative disorders such as cancers, leukaemias and other disorders associated with uncontrolled cellular proliferation such as psoriasis and restenosis. As defined herein, an anti-proliferative effect within the scope of the present invention may be demonstrated by the ability to inhibit cell proliferation in an in vitro whole cell assay, for example using any of the cell lines A2780, Mia-PaCa-2, A549, HT29 or Saos-2. Using such assays it may be determined whether a compound is anti-proliferative in the context of the present invention.

A preferred embodiment of the present invention therefore relates to the use of one or more compounds of formula Ia as defined above, or pharmaceutically acceptable salts thereof, in the preparation of a medicament for treating a proliferative disorder.

One preferred embodiment of the invention relates to the use of a compound of formula Ia, or a pharmaceutically acceptable salt thereof,

wherein R³, R⁴, R⁵, R⁶, R⁷, R⁸, R⁹, and R¹⁰ are each independently H, R¹¹ or R¹²;
R¹ and R² are each independently H, R¹¹ or R¹²; or R¹ and R² are linked to form a cyclic group together with the nitrogen to which they are attached, and wherein said cyclic group is optionally substituted with one or more R¹¹ or R¹² groups;
each R¹¹ is independently a hydrocarbyl group optionally substituted by one or more R¹² substituents;
each R¹² is independently selected from OR¹³, COR¹³, COOR¹³, CN, CONR¹³R¹⁴, NR¹³R¹⁴, SR¹³, SOR¹³, SO₂R¹³, SO₂OR¹³, SO₂NR¹³R¹⁴, an alicyclic group, halogen, CF₃, and NO₂; and
R¹³ and R¹⁴ are each independently H or (CH₂)ₙR¹⁵, where n is 0, 1, 2, or 3; and
each R¹⁵ is independently selected from alkyl, cycloalkyl, aryl, heteroaryl, aryl and an alicyclic group;
with the proviso that the compound is other than [4-(1H-indol-3-yl)-pyrimidin-2-yl]-[3-(1,1,2,2-tetrafluoroethoxyphenyl)]-amine;
in the preparation of a medicament for treating a proliferative disorder.

As used herein the phrase “preparation of a medicament” includes the use of a compound of the invention directly as the medicament in addition to its use in a screening programme for further therapeutic agents or in any stage of the manufacture of such a medicament.

Preferably, the proliferative disorder is a cancer or leukaemia.

The term proliferative disorder is used herein in a broad sense to include any disorder that requires control of the cell cycle, for example cardiovascular disorders such as restenosis, cardiomyopathy and myocardial infarction, auto-immune disorders such as glomerulonephritis and rheumatoid arthritis, dermatological disorders such as psoriasis, anti-inflammatory, anti-fungal, antiparasitic disorders such as malaria, emphysema, alopecia, and chronic obstructive pulmonary disorder. In these disorders, the compounds of the present invention may induce apoptosis or maintain stasis within the desired cells as required.

The compounds of the invention may inhibit any of the steps or stages in the cell cycle, for example, formation of the nuclear envelope, exit from the quiescent phase of the cell cycle (G0), G1 progression, chromosome decondensation, nuclear envelope breakdown, START, initiation of DNA replication, progression of DNA replication, termination of DNA replication, centrosome duplication, G2 progression, activation of mitotic or meiotic functions, chromosome condensation, centrosome separation, microtubule nucleation, spindle formation and function, interactions with microtubule motor proteins, chromatid separation and segregation, inactivation of mitotic functions, formation of contractile ring, and cytokinesis functions. In particular, the compounds of the invention may influence certain gene functions such as chromatin binding, formation of replication complexes, replication licensing, phosphorylation or other secondary modification activity, proteolytic degradation, microtubule binding, actin binding, septin binding, microtubule organising centre nucleation activity and binding to components of cell cycle signalling pathways.

In one embodiment of the invention, the compound of the invention is administered in an amount sufficient to inhibit at least one CDK enzyme. Preferably, the CDK enzyme is CDK1, CDK2, CDK3, CDK4, CDK6, CDK7, CDK8 and/or CDK9.

More preferably, the compound of the invention is administered in an amount sufficient to inhibit at least one of CDK2 and/or CDK4.

Another aspect of the invention relates to the use of a compound of formula Ib as defined above, or a pharmaceutically acceptable salt thereof, in the preparation of a medicament for treating a viral disorder. Preferably, the viral disorder is selected from human cytomegalovirus (HCMV), herpes simplex virus type 1 (HSV-1), human immunodeficiency virus type 1 (HIV-1) and varicella zoster virus (VZV).

In one preferred embodiment, the invention relates to the use of a compound of formula Ib, or a pharmaceutically acceptable salt thereof, as defined above,

wherein R³, R⁴, R⁵, R⁶, R⁷, R⁸, R⁹, and R¹⁰ are each independently H, R¹¹ or R¹²;

R¹ and R² are each independently H, R¹¹ or R¹²; or R¹ and R² are linked to form a cyclic group together with the nitrogen to which they are attached, and wherein said cyclic group is optionally substituted with one or more R¹¹ or R¹² groups;
each R¹¹ is independently a hydrocarbyl group optionally substituted by one or more R¹² substituents;
each R¹² is independently selected from OR¹³, COR¹³, COOR¹³, CN, CONR¹³R¹⁴, NR¹³R¹⁴, SR¹³, SOR¹³, SO₂R¹³, SO₂OR¹³, SO₂NR¹³R¹⁴, an alicyclic group, halogen, CF₃, and NO₂; and
R¹³ and R¹⁴ are each independently H or (CH₂)ₙR¹⁵, where n is 0, 1, 2, or 3; and
each R¹⁵ is independently selected from alkyl, cycloalkyl, aryl, heteroaryl, aryl and an alicyclic group;
in the preparation of a medicament for treating one or more of the following: a viral disorder; a CNS disorder; a stroke; a microbial infection; a fungal disorder; a parasitic disorder; an inflammatory disorder; a cardiovascular disorder; alopecia; and diabetes.

In a more preferred embodiment of the invention, the compound of the invention is administered in an amount sufficient to inhibit one or more of the host cell CDKs involved in viral replication, i.e. CDK2, CDK7, CDK8, and CDK9 [23].

As defined herein, an anti-viral effect within the scope of the present invention may be demonstrated by the ability to inhibit CDK2, CDK7, CDK8 or CDK9.

In a particularly preferred embodiment, the invention relates to the use of one or more compounds of the invention in the treatment of a viral disorder which is CDK dependent or sensitive. CDK dependent disorders are associated with an above normal level of activity of one or more CDK enzymes. Such disorders preferably associated with an abnormal level of activity of CDK2, CDK7, CDK8 and/or CDK9. A CDK sensitive disorder is a disorder in which an aberration in the CDK level is not the primary cause, but is downstream of the primary metabolic aberration. In such scenarios, CDK2, CDK7, CDK8 and/or CDK9 can be said to be part of the sensitive metabolic pathway and CDK inhibitors may therefore be active in treating such disorders.

A further aspect of the invention relates to a method of treating a CDK-dependent disorder, said method comprising administering to a subject in need thereof, a compound of formula Ia or Ib, or a pharmaceutically acceptable salt thereof, as defined above in an amount sufficient to inhibit a cyclin dependent kinase.

Preferably, the CDK-dependent disorder is a viral disorder or a proliferative disorder, more preferably cancer.

In one preferred embodiment, the compound of the invention is administered in an amount sufficient to inhibit FLT3. FLT3 is known to play an important role in the pathogenesis of acute myeloid leukemia [79]. Thus, in one particularly preferred embodiment, the proliferative disorder is acute myeloid leukemia.

Another aspect of the invention relates to a method of treating a FLT3-dependent disorder, said method comprising administering to a subject in need thereof, a compound of formula Ia or Ib, or a pharmaceutically acceptable salt thereof, in an amount sufficient to inhibit FLT3.

Another aspect of the invention relates to the use of a compound of formula Ib as defined above, or a pharmaceutically acceptable salt thereof, in the preparation of a medicament for treating diabetes.

In a particularly preferred embodiment, the diabetes is type II diabetes.

GSK3 is one of several protein kinases that phosphorylate glycogen synthase (GS). The stimulation of glycogen synthesis by insulin in skeletal muscle results from the dephosphorylation and activation of GS. GSK3's action on GS thus results in the latter's deactivation and thus suppression of the conversion of glucose into glycogen in muscles. Type II diabetes (non-insulin dependent diabetes mellitus) is a multi-factorial disease. Hyperglycaemia is due to insulin resistance in the liver, muscles, and other tissues, coupled with impaired secretion of insulin. Skeletal muscle is the main site for insulin-stimulated glucose uptake, there it is either removed from circulation or converted to glycogen. Muscle glycogen deposition is the main determinant in glucose homeostasis and type II diabetics have defective muscle glycogen storage. There is evidence that an increase in GSK3 activity is important in type II diabetes [24]. Furthermore, it has been demonstrated that GSK3 is over-expressed in muscle cells of type II diabetics and that an inverse correlation exists between skeletal muscle GSK3 activity and insulin action [25].

GSK3 inhibition is therefore of therapeutic significance in the treatment of diabetes, particularly type II, and diabetic neuropathy.

It is notable that GSK3 is known to phosphorylate many substrates other than GS, and is thus involved in the regulation of multiple biochemical pathways. For example, GSK is highly expressed in the central and peripheral nervous systems.

Preferably, the compound is administered in an amount sufficient to inhibit GSK, more preferably GSK3, more preferably still GSK3β.

Another aspect of the invention therefore relates to the use of a compound of formula Ib as defined above, or a pharmaceutically acceptable salt thereof, in the preparation of a medicament for treating a CNS disorder, for example neurodegenerative disorders.

Preferably, the CNS disorder is Alzheimer's disease.

Tau is a GSK-3 substrate which has been implicated in the etiology of Alzheimer's disease. In healthy nerve cells, Tau co-assembles with tubulin into microtubules. However, in Alzheimer's disease, tau forms large tangles of filaments, which disrupt the microtubule structures in the nerve cell, thereby impairing the transport of nutrients as well as the transmission of neuronal messages.

Without wishing to be bound by theory, it is believed that GSK3 inhibitors may be able to prevent and/or reverse the abnormal hyperphosphorylation of the microtubule-associated protein tau that is an invariant feature of Alzheimer's disease and a number of other neurodegenerative diseases, such as progressive supranuclear palsy, corticobasal degeneration and Pick's disease. Mutations in the tau gene cause inherited forms of fronto-temporal dementia, further underscoring the relevance of tau protein dysfunction for the neurodegenerative process [26].

Another aspect of the invention therefore relates to the use of a compound of formula Ib as defined above, or a pharmaceutically acceptable salt thereof, in the preparation of a medicament for treating bipolar disorder.

Yet another aspect of the invention relates the use of a compound of formula Ib as defined above, or a pharmaceutically acceptable salt thereof, in the preparation of a medicament for treating a stroke.

Reducing neuronal apoptosis is an important therapeutic goal in the context of head trauma, stroke, epilepsy, and motor neuron disease [27]. Therefore, GSK3 as a pro-apoptotic factor in neuronal cells makes this protein kinase an attractive therapeutic target for the design of inhibitory drugs to treat these diseases.

Yet another aspect of the invention relates the use of a compound of formula Ib as defined above, or a pharmaceutically acceptable salt thereof, in the preparation of a medicament for treating alopecia.

Hair growth is controlled by the Wnt signalling pathway, in particular Wnt-3. In tissue-culture model systems of the skin, the expression of non-degradable mutants of β-catenin leads to a dramatic increase in the population of putative stem cells, which have greater proliferative potential [28]. This population of stem cells expresses a higher level of non-cadherin-associated β-catenin [29], which may contribute to their high proliferative potential. Moreover, transgenic mice overexpressing a truncated β-catenin in the skin undergo de novo hair-follicle morphogenesis, which normally is only established during embryogenesis. The ectopic application of GSK3 inhibitors may therefore be therapeutically useful in the treatment of baldness and in restoring hair growth following chemotherapy-induced alopecia.

A further aspect of the invention relates to a method of treating a GSK3-dependent disorder, said method comprising administering to a subject in need thereof, a compound of formula Ib, or a pharmaceutically acceptable salt thereof, as defined above in an amount sufficient to inhibit GSK3.

Preferably, the GSK3-dependent disorder is diabetes.

Preferably, the compound of the invention, or pharmaceutically acceptable salt thereof, is administered in an amount sufficient to inhibit GSK3β.

In one embodiment of the invention, the compound of the invention is administered in an amount sufficient to inhibit at least one PLK enzyme.

The polo-like kinases (PLKs) constitute a family of serine/threonine protein kinases. Mitotic Drosophila melanogaster mutants at the polo locus display spindle abnormalities [30] and polo was found to encode a mitotic kinase [31]. In humans, there exist three closely related PLKs [32]. They contain a highly homologous amino-terminal catalytic kinase domain and their carboxyl termini contain two or three conserved regions, the polo boxes. The function of the polo boxes remains incompletely understood but they are implicated in the targeting of PLKs to subcellular compartments [33,34], mediation of interactions with other proteins [35], or may constitute part of an autoregulatory domain [36]. Furthermore, the polo box-dependent PLK1 activity is required for proper metaphase/anaphase transition and cytokinesis [37,38].

Studies have shown that human PLKs regulate some fundamental aspects of mitosis [39,40]. In particular, PLK1 activity is believed to be necessary for the functional maturation of centrosomes in late G2/early prophase and subsequent establishment of a bipolar spindle. Depletion of cellular PLK1 through the small interfering RNA (siRNA) technique has also confirmed that this protein is required for multiple mitotic processes and completion of cytokinesis [41].

In a more preferred embodiment of the invention, the compound of the invention is administered in an amount sufficient to inhibit PLK1.

Of the three human PLKs, PLK1 is the best characterized; it regulates a number of cell division cycle effects, including the onset of mitosis [42,43], DNA-damage checkpoint activation [44,45], regulation of the anaphase promoting complex [46-48], phosphorylation of the proteasome [49], and centrosome duplication and maturation [50].

Specifically, initiation of mitosis requires activation of M-phase promoting factor (MPF), the complex between the cyclin dependent kinase CDK1 and B-type cyclins [51]. The latter accumulate during the S and G2 phases of the cell cycle and promote the inhibitory phosphorylation of the MPF complex by WEE1, MIK1, and MYT1 kinases. At the end of the G2 phase, corresponding dephosphorylation by the dual-specificity phosphatase CDC25C triggers the activation of MPF [52]. In interphase, cyclin B localizes to the cytoplasm [53], it then becomes phosphorylated during prophase and this event causes nuclear translocation [54,55]. The nuclear accumulation of active MPF during prophase is thought to be important for initiating M-phase events [56]. However, nuclear MPF is kept inactive by WEE1 unless counteracted by CDC25C. The phosphatase CDC25C itself, localized to the cytoplasm during interphase, accumulates in the nucleus in prophase [57-59]. The nuclear entry of both cyclin B [60] and CDC25C [61] are promoted through phosphorylation by PLK1 [43]. This kinase is an important regulator of M-phase initiation.

In one particularly preferred embodiment, the compounds of the invention are ATP-antagonistic inhibitors of PLK1.

In the present context ATP antagonism refers to the ability of an inhibitor compound to diminish or prevent PLK catalytic activity, i.e. phosphotransfer from ATP to a macromolecular PLK substrate, by virtue of reversibly or irreversibly binding at the enzyme's active site in such a manner as to impair or abolish ATP binding.

In another preferred embodiment, the compound of the invention is administered in an amount sufficient to inhibit PLK2 and/or PLK3.

Mammalian PLK2 (also known as SNK) and PLK3 (also known as PRK and FNK) were originally shown to be immediate early gene products. PLK3 kinase activity appears to peak during late S and G2 phase. It is also activated during DNA damage checkpoint activation and severe oxidative stress. PLK3 also plays an important role in the regulation of microtubule dynamics and centrosome function in the cell and deregulated PLK3 expression results in cell cycle arrest and apoptosis [62]. PLK2 is the least well understood homologue of the three PLKs. Both PLK2 and PLK3 may have additional important post-mitotic functions [35].

A further aspect of the invention relates to a method of treating a PLK-dependent disorder, said method comprising administering to a subject in need thereof, a compound of formula Ib, or a pharmaceutically acceptable salt thereof, as defined above in an amount sufficient to inhibit PLK.

Preferably, the PLK-dependent disorder is a proliferative disorder, more preferably cancer.

Preferably, the compound of the invention, or pharmaceutically acceptable salt thereof, is administered in an amount sufficient to inhibit aurora kinase.

A further aspect of the invention relates to a method of treating an aurora kinase-dependent disorder, said method comprising administering to a subject in need thereof, a compound of formula Ib, or a pharmaceutically acceptable salt thereof, as defined above in an amount sufficient to inhibit aurora kinase.

Preferably, the aurora kinase dependent disorder is a viral disorder as defined above.

### Pharmaceutical Compositions

Another aspect of the invention relates to a pharmaceutical composition comprising one or more compounds of the invention as defined above admixed with one or more pharmaceutically acceptable diluents, excipients or carriers. Even though the compounds of the present invention (including their pharmaceutically acceptable salts, esters and pharmaceutically acceptable solvates) can be administered alone, they will generally be administered in admixture with a pharmaceutical carrier, excipient or diluent, particularly for human therapy. The pharmaceutical compositions may be for human or animal usage in human and veterinary medicine.

Examples of such suitable excipients for the various different forms of pharmaceutical compositions described herein may be found in the “Handbook of Pharmaceutical Excipients, 2^{nd} Edition, (1994), Edited by A Wade and P J Weller.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s).

Examples of suitable binders include starch, gelatin, natural sugars such as glucose; anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

### Salts/Esters

The compounds of the invention can be present as salts or esters, in particular pharmaceutically acceptable salts or esters.

Pharmaceutically acceptable salts of the compounds of the invention include suitable acid addition or base salts thereof. A review of suitable pharmaceutical salts may be found in Berge et al, J Pharm Sci, 1, 1-19 (1977). Salts are formed, for example with strong inorganic acids such as mineral acids, e.g. sulphuric acid, phosphoric acid or hydrohalic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen).

### Enantiomers/Tautomers

In all aspects of the present invention previously discussed, the invention includes, where appropriate all enantiomers and tautomers of compounds of the invention. The man skilled in the art will recognise compounds that possess an optical properties (one or more chiral carbon atoms) or tautomeric characteristics. The corresponding enantiomers and/or tautomers may be isolated/prepared by methods known in the art.

### Stereo and Geometric Isomers

Some of the compounds of the invention may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those agents, and mixtures thereof. The terms used in the claims encompass these forms, provided said forms retain the appropriate functional activity (though not necessarily to the same degree).

In particular, the compounds of the invention may exist in cis or trans forms, either in isolated form, or as mixtures thereof in any ratio. By way of example, where the compounds of the invention contain morpholinyl or piperidinyl substituents, the methyl groups on the morpholinyl and piperidinyl rings can be either cis or trans.

The present invention also includes all suitable isotopic variations of the agent or pharmaceutically acceptable salt thereof. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agent of the present invention and pharmaceutically acceptable salts thereof of this invention can generally be prepared by conventional procedures using appropriate isotopic variations of suitable reagents.

### Solvates

The present invention also includes the use of solvate forms of the compounds of the present invention The terms used in the claims encompass these forms.

### Polymorphs

The invention furthermore relates to the compounds of the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

### Prodrugs

The invention further includes the compounds of the present invention in prodrug form. Such prodrugs are generally compounds of the invention wherein one or more appropriate groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo. Examples of such modifications include ester (for example, any of those described above), wherein the reversion may be carried out be an esterase etc. Other such systems will be well known to those skilled in the art.

### Administration

The pharmaceutical compositions of the present invention may be adapted for oral, rectal, vaginal, parenteral, intramuscular, intraperitoneal, intraarterial, intrathecal, intrabronchial, subcutaneous, intradermal, intravenous, nasal, buccal or sublingual routes of administration.

For oral administration, particular use is made of compressed tablets, pills, tablets, gellules, drops, and capsules. Preferably, these compositions contain from 1 to 250 mg and more preferably from 10-100 mg, of active ingredient per dose.

Other forms of administration comprise solutions or emulsions which may be injected intravenously, intraarterially, intrathecally, subcutaneously, intradermally, intraperitoneally or intramuscularly, and which are prepared from sterile or sterilisable solutions. The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

Injectable forms may contain between 10-1000 mg, preferably between 10-250 mg, of active ingredient per dose.

Compositions may be formulated in unit dosage form, i.e., in the form of discrete portions containing a unit dose, or a multiple or sub-unit of a unit dose.

### Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

Depending upon the need, the agent may be administered at a dose of from 0.01 to 30 mg/kg body weight, such as from 0.1 to 10 mg/kg, more preferably from 0.1 to 1 mg/kg body weight.

In an exemplary embodiment, one or more doses of 10 to 150 mg/day will be administered to the patient.

### Combinations

In a particularly preferred embodiment, the one or more compounds of the invention are administered in combination with one or more other therapeutically active agents, for example, existing drugs available on the market. In such cases, the compounds of the invention may be administered consecutively, simultaneously or sequentially with the one or more other active agents.

By way of example, it is known that anticancer drugs in general are more effective when used in combination. In particular, combination therapy is desirable in order to avoid an overlap of major toxicities, mechanism of action and resistance mechanism(s). Furthermore, it is also desirable to administer most drugs at their maximum tolerated doses with minimum time intervals between such doses. The major advantages of combining chemotherapeutic drugs are that it may promote additive or possible synergistic effects through biochemical interactions and also may decrease the emergence of resistance in early tumor cells which would have been otherwise responsive to initial chemotherapy with a single agent. An example of the use of biochemical interactions in selecting drag combinations is demonstrated by the administration of leucovorin to increase the binding of an active intracellular metabolite of 5-fluorouracil to its target, thymidylate synthase, thus increasing its cytotoxic effects.

Numerous combinations are used in current treatments of cancer and leukemia A more extensive review of medical practices may be found in “Oncologic Therapies” edited by E. E. Vokes and H. M. Golomb, published by Springer.

Beneficial combinations may be suggested by studying the growth inhibitory activity of the test compounds with agents known or suspected of being valuable in the treatment of a particular cancer initially or cell lines derived from that cancer. This procedure can also be used to determine the order of administration of the agents, i.e. before, simultaneously, or after delivery. Such scheduling may be a feature of all the cycle acting agents identified herein.

### Assays

Another aspect of the invention relates to the use of a compound of formula Ib as defined above, or a pharmaceutically acceptable salt thereof, in an assay for identifying further candidate compounds capable of inhibiting one or more protein kinases.

Another aspect of the invention relates to the use of a compound of the invention, or a pharmaceutically acceptable salt thereof, in an assay for identifying further candidate compounds capable of inhibiting one or more cyclin dependent kinases, aurora kinase, GSK, FLT3 and PLK.

Preferably, the assay is a competitive binding assay.

More preferably, the competitive binding assay comprises contacting a compound of the invention with a protein kinase and a candidate compound and detecting any change in the interaction between the compound of the invention and the protein kinase.

One aspect of the invention relates to a process comprising the steps of:
(a) performing an assay method described hereinabove;
(b) identifying one or more ligands capable of binding to a ligand binding domain; and
(c) preparing a quantity of said one or more ligands.

Another aspect of the invention provides a process comprising the steps of:

(a) performing an assay method described hereinabove;
(b) identifying one or more ligands capable of binding to a ligand binding domain; and
(c) preparing a pharmaceutical composition comprising said one or more ligands.

Another aspect of the invention provides a process comprising the steps of:
(a) performing an assay method described hereinabove;
(b) identifying one or more ligands capable of binding to a ligand binding domain;
(c) modifying said one or more ligands capable of binding to a ligand binding domain;
(d) performing the assay method described hereinabove;
(e) optionally preparing a pharmaceutical composition comprising said one or more ligands.

The invention also relates to a ligand identified by the method described hereinabove.

Yet another aspect of the invention relates to a pharmaceutical composition comprising a ligand identified by the method described hereinabove.

Another aspect of the invention relates to the use of a ligand identified by the method described hereinabove in the preparation of a pharmaceutical composition for use in the treatment of proliferative disorders, viral disorders, a CNS disorder, stroke, alopecia and diabetes.

Preferably, said candidate compound is generated by conventional SAR modification of a compound of the invention.

As used herein, the term “conventional SAR modification” refers to standard methods known in the art for varying a given compound by way of chemical derivatisation.

The above methods may be used to screen for a ligand useful as an inhibitor of one or more protein kinases.

### Synthesis

Compounds of general formula I may be prepared by any suitable method known in the art. A convenient synthetic route is shown below:

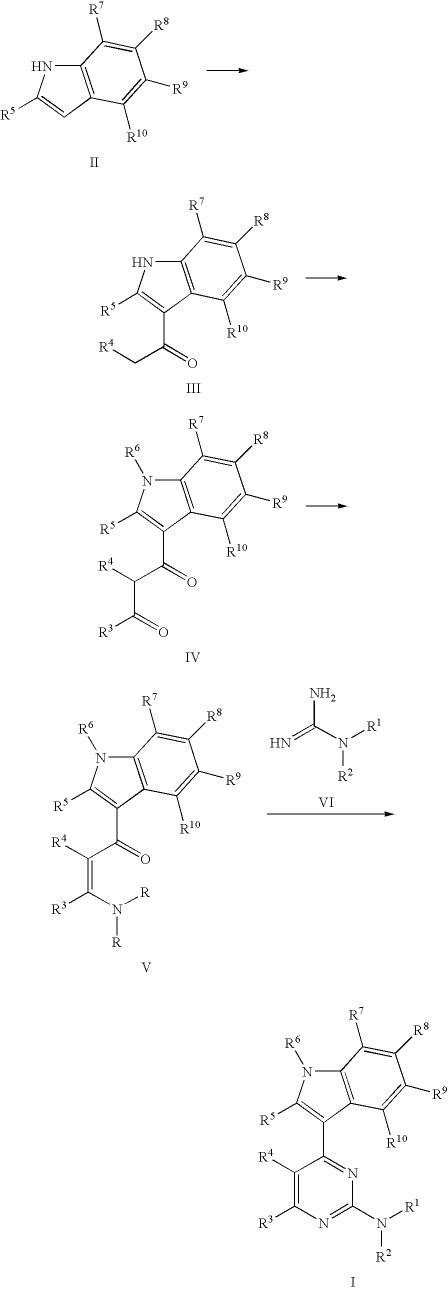

N-Unsubstituted 1-H-indoles II may be acylated with acid anhydride or acid halide derivatives of R⁴CH₂COOH at C-3 to afford the 3-acyl-1H-indole products III ([69], pp. 262-263). If R⁶ is other than H, this substituent is next introduced, followed by acylation with appropriate carbonyl compounds containing the group R³, to provide the intermediate 1,3-dicarbonyl compounds IV. These can be condensed directly with guanidines VI; alternatively they are first converted to the enaminones V, from which 4-(1H-Indol-3-yl)-pyrimidin-2-ylamines I can be obtained [70].

A further aspect of the invention therefore relates to a process for preparing a compound of formula Ib as defined above, said process comprising the steps of:

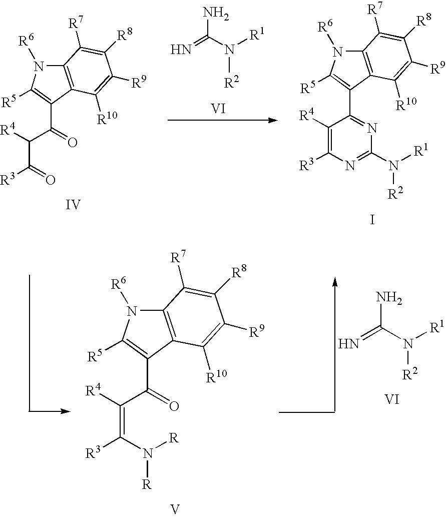

(a) condensing a compound of formula IV with a guanidine of formula VI to form a compound of formula I; or
(b) (i) converting a compound of formula IV to a compound of formula V; and
   (ii) condensing said compound of formula V with a guanidine of formula VI to form a compound of formula I.

Preferably, the compound of formula IV is prepared by acylating a compound of formula III

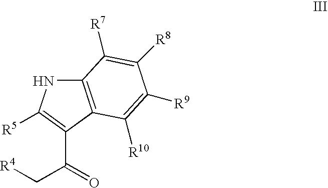

Preferably, the compound of formula III is prepared by acylating a compound of formula II with an acid anhydride or acid halide derivative of R⁴CH₂COOH

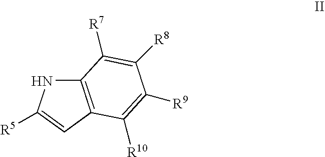

In one preferred embodiment, said compound of formula III is prepared by a process which comprises treating a compound of formula II as defined above with (i) zinc chloride and ethylmagnesium bromide, and (ii) acetyl chloride.

The present invention is further described by way of the following non-limiting examples.

### EXAMPLES

### Example 1

### General

NMR spectra were recorded using a Varian INOVA-500 instrument Chemical shifts are reported in parts per million relative to internal tetramethylsilane standard. Mass spectra were obtained using a Waters ZQ2000 single quadrupole mass spectrometer with electrospray ionization (ESI). Analytical and preparative RP-HPLC was performed using Vydac 218TP54 (250×4.6 mm) and 218TP1022 (250×22 mm) columns, respectively. Linear gradient elution using H₂O/MeCN systems (containing 0.1% CF₃COOH) at flow rates of 1 mL/min (analytical) and 9 mL/min (preparative) was performed. Purity was assessed by integration of chromatograms (λ=254 nm). Silica gel (EM Kieselgel 60, 0.040-0.063 mm, Merck) or ISOLUTE pre-packed columns (Jones Chromatography Ltd. UK) were used for flash chromatography.

The structures of selected compounds of the invention are shown in Table 1.

### Example 2

### 4-(1H-Indol-3-yl)-pyrimidin-2-ylamine (1)

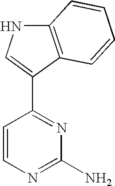

A mixture of 1-(1H-indol-3-yl)-ethanone (2 mmol, 3.18 g) in dimethoxymethyl-dimethyl-amine (60 mmol, 7.18 g, 8 mL) was heated under reflux for 16 h Excess dimethoxymethyl-dimethyl-amine was evaporated in vacuo to leave an orange residue of 3-dimethylamino-1-(1H-indol-3-yl)-propenone, which was used in the next reaction without further purification. A mixture of this material (5 mmol, 1.07 g) and guanidine carbonate (5 mmol, 0.94 g) in 2-methoxylethanol (20 mL) was heated at 125° C. for 22 h. The solvent was evaporated and the residue was purified by silica gel column chromatography (elution with 5:1 EtOAc/PE and then EtOAc). The fractions containing the desired product were combined and evaporated. The residue was recrystallised from MeOH to afford the pure title compound (0.80 g, 76%) as colourless crystals. ¹H-NMR (300 MHz, DMSO-d₆) δ: 6.43 (s, 1H, NH), 6.94 (d, 1H, J=5.3 Hz, pyrimidinyl-H), 7.14-7.27 (m, 2H, Ar—H), 7.49 (d, 1H, J=8.0 Hz, Ar—H), 8.10 (d, 1H, J=5.4 Hz, pyrimidinyl-H), 8.18 (s, 1H, indole C²—H), 8.59 (d, 1H, J=7.6 Hz, Ar—H).

### Example 3

### [4-(1H-Indol-3-yl)-pyrimidin-2-yl]-(3-nitro-phenyl)-amine (2)

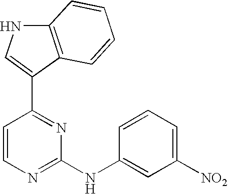

A mixture of 1-(1H-indol-3-yl)-ethanone (1.00 g, 6.28 mmol) and tert-butoxy-bis-(dimethyl-amino)-methane (1.5 mL, 1.16 mmol) was heated at 100° C. for 15 h. After cooling, and concentration under vacuum, the residue was treated with cold diethyl ether. The resulting yellow precipitate was filtered and dried to afford 3-dimethylamino-1-(1H-indol-3-yl)-propenone (0.40 g, 1.86 mmol. 29.7%). ¹H-NMR (500 MHz, DMSO-d₆) δ: 2.95 (6H, br. s, N(CH₃)₂), 5.74 (1H, d, C═CH, J=12.6 Hz), 7.08 (1H, dd, ArH, J=8.3, 8.3 Hz), 7.10 (1H, dd, ArH, J=7.8, 7.8 Hz), 7.38 (1H, d, ArH, J=7.8 Hz), 7.49 (1H, d, C═CH, J=12.6 Hz), 8.12 (1H, s, ArH), 8.26 (1H, d, ArH, J=7.8 Hz), 11.56 (1H, s, NH). ESI-MS: m/z=214.98 [M+H]⁺. C₁₃H₁₄N₂O requires 214.3; Anal. RP-HPLC (0-60% MeCN gradient) t_{R}=13.2 min (>98% purity).

3-Dimethylamino-1-(1H-indol-3-yl)-propenone (0.173 g, 0.8 mmol), N-(3-nitro-phenyl)-guanidine nitrate (0.197 g, 0.8 mmol) and potassium carbonate (0.139 g, 1.0 mmol) were combined in 2-methoxyethanol (4 mL) and heated at 115° C. for 22 h. After cooling, the inorganics were filtered off and the filtrate was concentrated to dryness. The crude product was purified by silica gel column chromatography. Pooling of the desired fractions, evaporation, and drying afforded the pure title compound (0.083 g, 0.25 mmol, 31%). ¹H-NMR (500 MHz, DMSO-d₆) δ 7.13 (1H, dd, ArH, J=8.3, 8.3 Hz), 7.20 (1H, dd, ArH, J=8.3, 8.3 Hz), 7.39 (1H, d, ArH, J=5.8 Hz), 7.48 (1H, d, ArH, J=8.3 Hz), 7.59 (1H, dd, ArH, J=8.3, 8.3 Hz), 7.80 (1H, dd, ArH, J=8.3, 2.3 Hz), 8.14 (1H, dd, ArH, J=8.3, 2.3 Hz), 8.40 1H, d, ArH, J=2.3 Hz), 8.42 (1H, d, ArH, J=5.8 Hz), 8.53 (1H, d, ArH, J=8.3 Hz), 8.97 (1H, s, ArH), 9.98 (1H, s, NH), 11.89 (1H, s, NH). ESI-MS m/z=331.94 [M+H]⁺, C₁₈H₁₃N₅O₂ requires 331.33. Anal. RP-HPLC (0-60% MeCN gradient) t_{R}=18.53 min (>98% purity).

### Example 4

The following compounds were prepared using procedures analogous to those described in Example 3 above:

### (4-Fluoro-phenyl)-[4-(1H-indol-3-yl)-pyrimidin-2-yl]-amine (3)

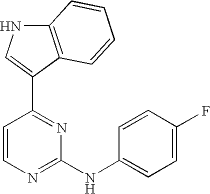

¹H-NMR (500 MHz, DMSO-d₆) δ: 7.13 (1H, dd, ArH, J=8.3, 8.3 Hz), 7.19 (1H, dd, ArH, J=8.3, 8.3 Hz), 7.19 (2H, d, ArH, J=8.3 Hz), 7.32 (1H, d, ArH, J=5.8 Hz), 7.46 (1H, d, ArH, J=8.3 Hz), 7.75 (2H, d, ArH, J=8.3 Hz), 8.27 (1H, d, ArH, J=5.8 Hz), 8.40 (1H, s, ArH), 8.47 (1H, d, ArH, J=8.3 Hz), 9.68 (1H, s, NM, 11.94 (1H, s, NH). ESI-MS m/z=305.05 [M+H]⁺, C₁₈H₁₃FN₄ requires 304.32. Anal. RP-HPLC (0-60% MeCN gradient) t_{R}=18.24 min (>98% purity).

### [4-(1H-Indol-3-yl)-pyrimidin-2-yl]-(6-methoxy-pyridin-3-yl)-amine (4)

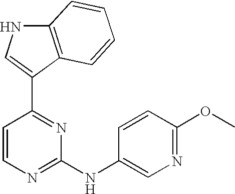

¹H-NMR (500 MHz, CD₃OD) δ: 3.96 (3H, s, CH₃), 6.90 (1H, d, J=9.0 Hz, ArH), 7.16 (1H, t, J=8.5 Hz, ArA), 7.23 (1H, t, J=8.5 Hz, ArH), 7.31 (1H, d, J=7.5 Hz, ArH), 7.46 (1H, d, J=5.5 Hz, pyrimidinyl-H), 7.98 (1H, m, ArH), 8.12 (1H, d, J=5.5 Hz, pyrimidinyl-H), 8.28 (2H, m, ArH), 8.39 (1H, s, ArH). ESI-MS m/z=317.99 [M+H]⁺, C₁₈H₁₅N₅O requires 317.34. Anal. RP-HPLC (10-70% MeCN gradient) t_{R}=12.88 min (purity>98%).

### [4-(1H-Indol-3-yl)-pyrimidin-2-yl]-(4-morpholin-4-yl-phenyl)-amine (5)

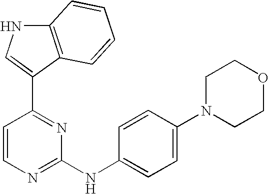

3-Dimethylamino-1-(1H-indol-3-yl)-propenone (0.10 g, 0.46 mmol), N-(4-morpholin-4-yl-phenyl)-guanidine nitrate (0.11 g, 0.46 mmol) and potassium carbonate (64 mg, 0.46 mmol) were combined in 2-methoxyethanol (4 mL) and the mixture was heated under microwave irradiation at 180° C. for 20 min. After cooling, the reaction product was precipitated by addition of water (25 mL), and was collected by filtration. The product was purified by silica gel chromatography. Pooling of the desired fractions afforded the pure target compound (40 mg, 23%). ¹H-NMR (500 MHz, CD₃OD) δ: 11.73 (1H, br. s, NH); 9.12 (1H, s, ArH); 8.57 (1H, d, ArH, J=7.8 Hz); 8.26 (2H, d, ArH, J=5.4 Hz); 7.65 (2H, d, ArH, J=8.8 Hz); 7.45 (1H, d, ArH, J=7.8 Hz); 7.18 (2H, m, ArH); 7.12 (1H, t, ArH, J=7.8 Hz); 6.92 (2H, d, ArH, J=9.3 Hz); 3.75 (4H, t, ArH, J=4.39 Hz); 3.05 (1H, d, ArH, J=5.15 Hz). ESI-MS m/z=371.98 [M+H]⁺, C₂₂H₂₁N₅O requires 371.44. Anal. RP-HPLC (10-70% MeCN gradient) t_{R}=12.02 min (purity>98%).

### Example 5

Compounds (6) to (31) were synthesised by analogous methods and in accordance with the protocols set forth below.

### 3-Acetylation of Substituted Indoles

[Concise Synthesis and Structure-Activity Relationships of Combrestatin A-4 Analogues: 1- and 3-Aroylindoles as Novel Classes of Potent Antitubulin Agents. J. Med. Chem., 2004, Vol. 47, No. 17, 4247-4257]

To a mixture of substituted indole (2.03 mmol) and anhydrous zinc chloride (560 mg, 4.07 mmol) in dry dichloromethane (15 ml), was added ethylmagnesium bromide (2.7 ml, 1.0M solution in THF) over 10 min at room temperature. After stirring for 1 h, acetyl chloride (239 mg, 217 μl, 3.05 mmol) was added dropwise over 5 min. After stirring for another 1 h, aluminium chloride (270 mg, 2.03 mmol) was added and the resultant mixture stirred for 5 h. Water was added (15 ml) and extracted with dichloromethane (20 ml). The organic layer was dried (MgSO₄) and evaporated to give a red solid. Chromatography (2:1 petroleum ether-ethyl acetate) gave the desired 3-acetyl indole as a colourless solid.

### Formation of 1-Acylindole Pyrimidines: Compounds (18) and (19)

Compound (5) (50 mg, 0.135 mmol) was dissolved in dry dimethylformamide (10 ml) and sodium hydride (4 mg, 1.2 eq) added. When evolution of hydrogen had ceased, acid chloride (1.2 eq) was added and the mixture stirred at room temperature for a further 4 h. Solvent removed in Genevac and the crude product suspended in MeOH (2 ml) and absorbed on SPE column. Chromatography (95:5 ethyl acetate-MeOH) gave the desired product as a yellow solid.

Preparation of indoles modified at the 2-position (Compound 22), the 6-position (Compounds 24 and 26), and the 7-position (Compounds 21, 23 and 25), and other indoles modified at the 1-position (Compound 16). These indoles were obtained as staring materials.

### 4-(1H-indol-3-yl)-N-(4-acetylpiperazin-1-ylphenyl)pyrimidin-2-amine (6)

Anal. RP-HPLC: t_{R} 13.01 min. (0-60% MeCN).

¹H NMR (DMSO-d₆, 500 MHz): δ_{H} 2.05 (3H, s, CH₃), 3.03 (2H, dd, J 5.0, 2×CHH), 3.09 (2H, dd, J 5.0, 2×CHH), 3.58 (4H, m, incl. J 5.0, 2×CH₂), 6.95 (2H, d, J 9.0, 2×Ar—H), 7.13 (1H, dd, J 7.5, Ar—H), 7.14-7.20 (2H, m, pyrim-H and Ar—H), 7.46 (1H, dd, J 7.5, Ar—H), 7.67 (2H, d, J 9.0, 2×Ar—H), 8.26 (2H, m, incl. J 5.0, pyrim-H and Ar—H), 8.57 (1H, dd, J 7.5, Ar—H), 9.14 (1H, s, NH) and 11.74 (1H, s, NH).

MS (ESI⁺) m/z 412.97 [M+H]⁺ (C₂₄H₂₄N₆O requires 412.49).

### 4-(1H-indol-3-yl)-N-(4-piperazin-1-ylphenyl)pyrimidin-2-amine (7)

Anal. RP-HPLC: t_{R} 9.48 min. (10-70% MeCN).

¹H NMR (DMSO-d₆, 500 MHz): δ_{H} 2.85 (4H, dd, J 5.0, 2×CH₂), 2.99 (41, dd, J 5.0, 2×CH₂), 6.89 (2H, d, J 8.5, 2×Ar—H), 7.12 (1H, dd, J 7.5, Ar—H), 7.16-7.20 (2H, m, pyrim-H and Ar—H), 7.45 (1H, dd, J 7.5, Ar—H), 7.63 (2H, d, J 8.5, 2×Ar—H), 8.26 (2H, m, incl. J 5.0, pyrim-H and Ar—H), 8.57 (1H, dd, J 7.5, Ar—H), 9.09 (1H, s, NH) and 11.73 (1H, s, NH).

MS (ESI⁺) m/z 371.02 [M+H]⁺ (C₂₂H₂₂N₆ requires 370.45).

### 4-(1H-indol-3-yl)-N-(4-benzylpiperazin-1-ylphenyl)pyrimidin-2-amine (8)

Anal. RP-HPLC: t_{R} 12.31 min. (10-70% MeCN).

¹H NMR (DMSO-d₆, 500 MHz): δ_{H} 2.51 (4H, dd, J 5.0, 2×CH₂), 3.09 (4H, dd, J 5.0, 2×CH₂), 3.53 (2H, s, CH₂), 6.91 (2H, d, J 9.0, 2×Ar—H), 7.12 (1H, dd, J 8.0, Ar—H), 7.16-7.20 (2×, m, pyrim-H and Ar—H), 7.26 (1H, dd, J 9.0, 4.0, Ar—H), 7.33-7.35 (4H, m, 4×Ar—H), 7.45 (1H, d, J 8.0, Ar—H), 7.63 (2H, d, J 9.0, 2×Ar—H), 8.25 (2H, m, incl. J 5.0, pyrim-H and Ar—H), 8.56 (1H, dd, J 8.0, 1.0, Ar—H), 9.09 (1H, s, NH) and 11.73 (1H, s, NH).

MS (ESI⁺) m/z 460.93 [M]⁺ (C₂₉H₂₈N₆ requires 460.57).

### 4-(1H-indol-3-yl)-N-(2,6-dimethylmorpholin-4-ylphenyl)pyrimidin-2-amine (9)

Two batches were made. Batch 01 is 20:1 cis:trans. Batch 02 is 1:1 cis:trans.

This compound was the result of a cyclisation with a guanidine containing a 4:1 cis:trans ratio of diastereoisomers. The early fractions from Prep-HPLC contained a 20:1 cis:trans mixture (Batch 01) and later fractions had a 1:1 mixture (Batch 02). The assay data refers to Batch 01.

Anal. RP-HPLC: t_{R} 14.05 min. (10-70% MeCN).

¹H NMR (DMSO-d₆, 500 MHz): δ_{H} 1.16 (6H, d, J 6.5, 2×CH₃), 2.22 (2H, dd, J 11.0, 2×CHH), 3.50 (2H, dd, J 11.0, 2×CHH), 3.71 (2H, m, incl. J 11.0, 6.5, 2×CHCH₃), 6.92 (2H, dd, J 8.0, 2×Ar—H), 7.12-7.15 (1H, m, Ar—H), 7.17-7.19 (2H, m, pyrim-H and Ar—H), 7.46 (1H, d, J 8.0, Ar—H), 7.65 (2H, d, J 8.0, 2×Ar—H), 8.26 (2H, m, incl. J 5.0, pyrim-H and Ar—H), 8.56 (1H, d, J 8.0, Ar—H), 9.10 (1H, s, NH) and 11.73 (1H, s, NH).

MS (ESI⁺) m/z 399.98 [M+H]⁺ (C₂₄H₂₅N₅O requires 399.49).

### N′-[4-(1H-indol-3-yl)pyrimidin-2-yl]-N,N-dimethylbenzene-1,4-diamine (10)

Anal. RP-HPLC: t_{R} 9.45 min. (10-70% MeCN).

¹H NMR (DMSO-d₆, 500 MHz): δ_{H} 2.86 (6H, s, 2×CH₃), 6.74 (2H, d, J 7.0, 2×Ar—H), 7.11 (1H, dd, J 8.0, Ar—H), 7.14-7.20 (2H, m, pyrim-H and Ar—H), 7.45 (1H, d, J 8.0, Ar—H), 7.58 (2H, d, J 7.0, 2×Ar—H), 8.23-8.26 (2H, m, incl. J 5.0, pyrim-H and Ar—H), 8.56 (1H, d, J 8.0, Ar—H), 8.99 (1H, s, NH) and 11.71 (1H, s, NH).

MS (ESI⁺) m/z 330.05 [M+H]⁺ (C₂₀H₁₉N₅ requires 329.40).

### 4-(1H-indol-3-yl)-N-(2-methyl-4-morpholin-4-ylphenyl)pyrimidin-2-amine (11)

Anal. RP-HPLC: t_{R} 12.48 min. (10-70% MeCN).

¹H NMR (DMSO-d₆, 500 MHz): δ_{H} 2.19 (3H, s, CH₃), 3.11 (4H, dd, J 5.0, 2×CH₂), 3.77 (4H, dd, J 5.0, 2×CH₂), 6.81 (1H, dd, J 9.0, 2.5, Ar—H), 6.86 (1H, d, J 2.5, Ar—H), 6.95 (1H, dd, J 8.0, Ar—H), 7.09-7.14 (2H, m, pyrim-H and Ar—H), 7.27 (1H, d, J 9.0, Ar—H), 7.39 (1H, d, J 8.0, Ar—H), 8.16 (1H, d, J 5.5, pyrim-H), 8.20 (211 br s, 2×Ar—H), 8.41 (1H, s, NH) and 11.65 (1H, s, NH).

MS (ESI⁺) m/z 385.98 [M+H]⁺ (C₂₃H₂₃N₅O requires 385.46).

### 4-(1H-indol-3-yl)-N-(3,4,5-trimethoxyphenyl)pyrimidin-2-amine (12)

Anal. RP-HPLC: t_{R} 13.60 min. (10-70% MeCN).

¹H NMR (DMSO-d₆, 500 MHz): δ_{H} 3.64 (3H, s, CH₃), 3.75 (6H, s,2×CH₃), 7.13 (1H, dd, J 7.5, Ar—H), 7.19 (1H, dd, J 7.5, Ar—H), 7.23-7.26 (3H, m, pyrim-H and 2×Ar—H), 7.46 (1H, d, J 7.5, Ar—H), 8.30 (1H, s, Ar—H), 8.32 (1H, d, J 5.0, pyrim-H), 8.58 (1H, d, J 7.5, Ar—H), 9.24 (1H, s, NH) and 11.76 (1H, s, NH).

MS (ESI⁺) m/z 376.97 [M+H]⁺ (C₂₁H₂₀N₄O₃ requires 376.41).

### 4-(1H-indol-3-yl)-N-(3-methoxyl-4-morpholin-4-ylphenyl)pyrimidin-2-amine (13)

Anal. RP-HPLC: t_{R} 10.67 min. (10-70% MeCN).

¹H NMR (DMSO-d₆, 500 MHz): δ_{H} 2.93 (4H, dd, J 5.0, 2×CH₂), 3.72 (4H, dd, J 5.0, 2×CH₂), 3.76 (3H, s, CH₃), 6.85 (1H, d, J 9.0, Ar—H), 7.12 (1H, dd, J 7.5, Ar—H), 7.17-7.23 (2H, m, pyrim-H and Ar—H), 7.37 (1H, dd, J 9.0, 2.0, Ar—H), 7.45-7.49 (2H, m, 2×Ar—H), 8.28 (1H, s, Ar—H), 8.30 (1H, d, J 5.0, pyrim-H), 8.58 (1H, d, J 7.5, Ar—H), 9.20 (1H, s, NH) and 11.76 (1H, s, NH).

MS (ESI⁺) m/z 402.03 [M]⁺ (C₂₃H₂₃N₅O₂ requires 401.46).

### N-(3,5-dimethoxyphenyl)-4-(1H-indol-3-yl)pyrimidin-2-amine (14)

Anal. RP-HPLC: t_{R} 15.06 min. (10-70% MeCN).

¹H NMR (DMSO-d₆, 500 MHz): δ_{H} 3.73 (6H, s, 2×CH₃), 6.13 (1H, s, Ar—H), 7.14 (3H, s, 3×Ar—H, 7.19 (1H, dd, J 8.0, Ar—H), 7.28 (1H, d, J 5.5, pyrim-H), 7.46 (1H, d, J 8.0, Ar—H), 8.30 (1H, s, Ar—H), 8.34 (1H, d, J 5.5, pyrim-H), 8.62 (1H, dd, J 8.0, Ar—H), 9.33 (1H, s, NH) and 11.79 (1H, s, NH).

MS (ESI⁺) m/z 344.90 [M]⁻ (C₂₀H₁₈N₄O₂ requires 346.38).

### 4-(1-methyl-1H-indol-3-yl)-N-(4-morpholin-4-ylphenyl(pyrimidin-2-amine (15)

Anal. RP-HPLC: t_{R} 13.64 min. (10-70% MeCN).

¹H NMR (DMSO d₆, 500 MHz): δ_{H} 3.06 (4H, dd, J 5.0, 2×CH₂), 3.75 (4H, dd, J 5.0, 2×CH₂), 3.88 (3H, s, CH₃), 6.93 (2H, d, J 8.0, 2×Ar—H), 7.12 (1H, d, J 5.5, pyrim-H), 7.18 (1H, dd, J 7.5, Ar—H), 7.26 (1H, dd, J 7.5, Ar—H), 7.52 (1H, d, J 7.5, Ar—H), 7.65 (2H, d, J 8.0, 2×Ar—H), 8.26-8.28 (2H, m, pyrim-H and Ar—H), 8.58 (1H, d, J 7.5, Ar—H) and 9.13 (1H, 9, NH).

MS (ESI⁺) m/z 385.98 [M+H]⁺ (C₂₃H₂₃N₅O requires 385.46).

### 4-(1-methyl-1H-indol-3-yl)-N-(4-acetylpiperazine-1-ylphenyl)pyrimidin-2-amine (16)

Anal. RP-HPLC: t_{R} 12.95 min. (10-70% MeCN).

¹H NMR (DMSO-d₆, 500 MHz): δ_{H} 2.04 (3H, s, CH₃), 3.02 (2H, dd, J 5.0, 2×CHH), 3.09 (2H, dd, J 5.0, 2×CHH), 3.59 (4H, q, J 5.0, 2×CH₂), 3.88 (3H, s, CH₃), 6.95 (2H, d, J 9.5, 2×Ar—H), 7.12 (1H, d, J 5.5, pyrim-H), 7.18 (1H, dd, J 7.5, Ar—H), 7.26 (1H, dd, J 7.5, Ar—H), 7.52 (1H, d, J 7.5, Ar—H), 7.66 (2H, d, J 9.5, 2×Ar—H), 8.26-8.28 (2H, m, incl. J 5.5, pyrim-H and Ar—H), 8.58 (1H, d, J 7.5, Ar—H) and 9.15 (1H, s, NH).

MS (ESI⁺) m/z 426.91 [M+H]⁺ (C₂₅H₂₆N₆O requires 426.51).

### N-1,3-benzodioxol-5-yl-4-(1H-indol-3-yl)pyrimidin-2-amine (17)

Anal. RP-HPLC: t_{R} 12.95 min. (10-70% MeCN).

¹H NMR (DMSO-d₆, 500 MHz): δ_{H} 5.99 (2H, s, CH₂), 6.86 (1H, d, J 8.5, Ar—H), 7.13 (1H, dd, J 7.5, Ar—H), 7.17-7.20 (2H, m, 2×Ar—H), 7.22 (1H, d, J 5.5, pyrim-H), 7.46 (1H, d, 7.5, Ar—H), 7.55 (1H, s, Ar—H), 8.28 (1H, s, Ar—H), 8.29 (1H, d, J 5.5, pyrim-H), 8.57 (1H, d, J 7.5, Ar—H), 9.25 (1H, s, NH) and 11.76 (1H, s, NH).

MS (ESI⁺) m/z 331.01 [M+H]⁺ (C₁₉H₁₄N₄O₂ requires 330.34).

### 4-[1-(cyclopropylcarbonyl)-1H-indol-3-yl]-N-(4-morpholin-4-ylphenyl)pyrimidin-2-amine (18)

Anal. RP-HPLC: t_{R} 14.35 min. (10-70% MeCN).

¹H NMR (DMSO-d₆, 500 MHz): δ_{H} 1.14-1.20 (4H, m, 2×cycloprop-CH₂), 2.87-2.91 (1H, m, cycloprop-CH), 3.04-3.08 (4H, m, 2×CH₂), 3.74-3.77 (4H, m, 2×CH₂), 6.92-6.95 (2H, m, 2×Ar—H), 7.34 (1H, dd, J 7.5, Ar—H), 7.40 (1H, dd, J 7.5, Ar—H), 7.44 (1H, d, J 5.5, pyrim-H), 7.61-7.65 (2H, m, 2×Ar—H), 8.40 (1H, d, J 8.5, Ar—H), 8.43 (1H, d, J 5.5, pyrim-H), 8.72 (1H, dd, J 7.5, Ar—H), 9.10 (1H, s, Ar—H) and 9.35 (1H, s, NH).

MS (ESI⁺) m/z 439.89 [M]⁺ (C₂₆H₂₅N₅O₂ requires 439.51).

### 4-(1-acetyl-1H-indol-3-yl)-N-(4-morpholin-4-ylphenyl)pyrimidin-2-amine (19)

Anal. RP-HPLC: t_{R} 14.44 min. (10-70% MeCN).

¹H NMR (DMSO-d₆, 500 MHz): δ_{H} 2.76 (3H, s, CH₃), 3.06 (4H, dd, J 5.0, 2×CH₂), 3.75 (4H, dd, J 5.0, 2×CH₂), 6.94 (2H, d, J 8.5, 2×Ar—H), 7.34 (1H, dd, J 8.5, Ar—H), 7.38-7.42 (2H, m, incl. J 5.0, pyrim-H and Ar—H), 7.63 (2H, d, J 8.5, 2×Ar—H), 8.40 (1H, d, J 8.5, Ar—H), 8.43 (1H, d, J 5.0, pyrim-H), 8.65-8.67 (1H, m, Ar—H), 8.73 (1H, s, Ar—H) and 9.35 (1H, s, NH).

MS (ESI⁺) m/z 414.95 [M+H]⁺ (C₂₄H₂₃N₅O₂ requires 413.47).

### 4-(1H-indol-3-yl)-N-(4-methylpiperazin-1-ylphenyl)pyrimidin-2-amine (20)

Anal. RP-HPLC: t_{R} 9.89 min. (10-70% MeCN).

¹H NMR (DMSO-d₆, 500 MHz): δ_{H} 2.23 (3H, s, CH₃), 2.47 (4H, dd, J 5.0, 2×CH₂), 3.08 (4H, dd, J 5.0, 2×CH₂), 6.91 (2H, d, J 9.0, 2×Ar—H), 7.12 (1H, dd, J 8.0, Ar—H), 7.16-7.20 (2H, m, incl. J 5.0, pyrim-H and Ar—H), 7.45 (1H, d, J 8.0, Ar—H), 7.63 (2H, d, J 9.0, 2×Ar—H), 8.26 (1H, d, J 5.0, pyrim-H), 8.27 (1H, s, Ar—H), 8.75 (1H, d, J 8.0, Ar—H), 9.10 (1H, s, Ar—H) and 11.73 (1H, s, NH).

MS (ESI⁺) m/z 385.02 [M+H]⁺ (C₂₃H₂₄N₆ requires 384.48).

### 4-(7-methoxy-1H-indol-3-yl)-N-(4-acetylpiperazin-1-ylphenyl)pyrimidin-2-amine (21)

Anal. RP-HPLC: t_{R} 9.89 min. (10-70% MeCN).

¹H NMR (DMSO-d₆, 500 MHz): δ_{H} 2.05 (3H, s, CH₃), 3.02 (2H, dd, J 5.0, 2×CH, 3.09 (2H, dd, J 5.0, 2×CHH), 3.59 (4H, dd, J 5.0, 2×CH₂), 3.94 (3H, s, CH₃), 6.76 (1H, d, J 7.5, Ar—H), 6.94 (2H, d, J 9.0, 2×Ar—H), 7.05 (1H, dd, J 7.5, Ar—H), 7.20 (1H, d, J 5.0, pyrim-H), 7.66 (2H, d, J 9.0, 2×Ar—H), 8.14-8.16 (2H, m, 2×Ar—H), 8.26 (1H, d, J 5.0, pyrim-H), 9.12 (1H, s, Ar—H) and 11.87 (1H, s, NH).

MS (ESI⁺) m/z 443.39 [M+H]⁺ (C₂₅H₂₆N₆O₂ requires 442.51).

### 4-(2-methyl-1H-indol-3-yl)-N-(4-acetylpiperazin-1-ylphenyl)]pyrimidin-2-amine (22)

Anal. RP-HPLC: t_{R} 12.09 min. (10-70% MeCN).

¹H NMR (DMSO-d₆, 500 MHz): δ_{H} 2.04 (3H, s, CH₃), 2.99 (3H, s, CH₃), 3.00 (2H, dd, J 5.0, 2×CHH), 3.07 (2H, dd, J 5.0, 2×CHH), 3.58 (4H, dd, J 5.0, 2×CH₂), 6.91 (2H, d, J 9.0, 2×Ar—H), 6.98 (1H, d, J 5.0, pyrim-H), 7.06 (1H, dd, J 8.0, Ar—H), 7.10 (1H, dd, J 8.0, Ar—H), 7.36 (1H, d, J 8.0, Ar—H), 7.67 (2H, d, J 9.0, 2×Ar—H), 8.15 (1H, d, J 8.0, Ar—H), 8.34 (1H, d, J 5.0, pyrim-H), 9.14 (1H, s, Ar—H) and 11.56 (1H, s, NH).

MS (ESI⁺) m/z 427.39 [M+H]⁺ (C₂₅H₂₆N₆O requires 426.51).

### 4-(7-methyl-1H-indol-3-yl)-N-(4-acetylpiperazin-1-ylphenyl)]pyrimidin-2-amine (23)

Anal. RP-HPLC: t_{R} 12.00 min. (10-70% MeCN).

¹H NMR (DMSO-d₆, 500 MHz): δ_{H} 1.99 (3H, s, CH₃), 2.48 (3H, s, CH₃), 2.98 (2H, dd, J 5.0, 2×CHH), 3.05 (2H, dd, J 5.0, 2×CHH), 3.55 (4H, dd, J 5.0, 2×CH₂), 6.91 (2H, d, J 9.0, 2×Ar—H), 6.94 (1H, d, J 7.5, Ar—H), 7.00 (1H, dd, J 7.5, Ar—H), 7.18 (1H, d, J 5.0, pyrim-H), 7.62 (2H, d, J 9.0, 2×Ar—H), 8.21-8.23 (2H, m, incl. J 5.0, pyrim-H and Ar—H), 8.37 (1H, d, J 7.5, Ar—H), 9.09 (1H, s, Ar—H) and 11.67 (1H, s, NH).

MS (ESI⁺) m/z 427.38 [M+H]⁺ (C₂₅H₂₆N₆O requires 426.51).

### 4-(6-methoxy-4H-indol-3-yl)-N-(4-acetylpiperazin-1-ylphenyl)]pyrimidin-2-amine (24)

Anal. RP-HPLC: t_{R} 11.27 min. (10-70% MeCN).

¹H NMR (DMSO-d₆, 500 MHz): δ_{H} 2.01 (3H, s, CH₃), 2.99 (2H, dd, J 5.0, 2×CHH), 3.06 (2H, dd, J 5.0, 2×CHH), 3.55 (4H, dd, J 5.0, 2×CH₂), 3.76 (3H, s, CH₃), 6.73 (1N, dd, J 9.0, 2.0, Ar—H), 6.90-6.92 (3H, m, incl. J 9.0, 3×Ar—H), 7.12 (1H, d, J 5.0, pyrim-H), 7.62 (2H, d, J 9.0, 2×Ar—H), 8.10 (1H, d, J 2.0 Ar—H), 8.21 (1H, d, J 5.0, pyrim-H), 8.40 (1H, d, J 9.0, Ar—H), 9.08 (1H, s, Ar—H) and 11.50 (1H, s, NH).

MS (ESI⁺) m/z 443.28 [M+H]⁺ (C₂₅H₂₆N₆O₂ requires 442.51).

### 4-(7-chloro-(H-indol-3-yl)-N-(4-acetylpiperazin-1-ylphenyl)]pyrimidin-2-amine (25)

Anal. RP-HPLC: t_{R} 11.27 min. (10-70% MeCN).

¹H NMR (DMSO-d₆, 500 MHz): δ_{H} 2.05 (3H, s, CH₃), 3.03 (2H, dd, J 5.0, 2×CHH), 3.10 (2H, dd, J 5.0, 2×CHH), 3.60 (4H, dd, J 5.0, 2×CH₂), 6.95 (2H, d, J 9.5, 2×Ar—H), 7.13 (1H, dd, J 8.0, Ar—H), 7.25-7.29 (2H, m, incl. J 5.0, pyrim-H and Ar—H), 7.65 (2H, d, J 9.5, 2×Ar—H), 8.29 (1H, d, J 5.0, pyrim-H), 8.36 (1H, s, Ar—H), 8.59 (1H, d, J 8.0, Ar—H), 9.19 (1H, s, Ar—H) and 12.11 (1H, s, NH).

MS (ESI⁺) m/z 447.37 [M+H]⁺ (C₂₄H₂₃N₆OCl requires 446.93).

### 4-(6-fluoro-(H-indol-3-yl)-N-(4-acetylpiperazin-1-ylphenyl)]pyrimidin-2-amine (26)

Anal. RP-HPLC: t_{R} 11.27 min. (10-70% MeCN).

¹H NMR (DMSO-d₆, 500 MHz): δ_{H} 2.05 (3H, s, CH₃), 3.03 (2H, dd, J 5.0, 2×CHH), 3.10 (2H, dd, J 5.0, 2×CHH), 3.59 (4H, dd, J 5.0, 2×CH₂), 6.94-7.00 (3H, m, incl. J 10.0, 9.5, 3×Ar—H), 7.19 (1H, d, J 5.5, pyrim-H), 7.24 (1H, dd, J 10.0, 1.0, Ar—H), 7.64 (2H, dd, J 9.5, 3.0, 2×Ar—H), 8.27 (1H, d, J 5.5, pyrim-H), 8.29 (1H, d, J 3.0, Ar—H), 8.58-8.62 (1H, m, Ar—H), 9.17 (1H, s, Ar—H) and 11.79 (1H, s, NH).

MS (ESI⁺) m/z 431.34 [M+H]⁺ (C₂₄H₂₃N₆OF requires 430.48).

### 4-(1H-indol-3-yl)-N-[(4-acetylpiperazin-1-yl)-3-methylphenyl]pyrimidin-2-amine (27)

Anal. RP-HPLC: t_{R} 13.44 min. (10-70% MeCN).

¹H NMR (DMSO-d₆, 500 MHz): δ_{H} 2.05 (3H, s, CH₃), 2.31 (3H, s, CH₃), 2.76 (2H, dd, J 5.0, 2×CHH), 2.83 (2H, dd, J 5.0, 2×CHH), 3.58 (4H, dd, J 5.0, 2×CH₂), 6.99 (1H, d, J 8.5, Ar—H), 7.13 (1H, dd, J 7.5, Ar—H), 7.17-7.23 (2H, m, incl. J 7.5, 5.5, pyrim-H and Ar—H), 7.46 (1H, d, J 7.5, Ar—H), 7.50 (1H, dd, J 8.5, 2.5, Ar—H), 7.73 (1H, d, J 2.5, Ar—H), 8.28 (2H, m, incl. J 5.5, pyrim-H and Ar—H), 8.59 (1H, d, J 7.5, Ar—H), 9.19 (1H, s, Ar—H) and 11.76 (1H, s, NH).

MS (ESI⁺) m/z 427.30 [M+H]⁺ (C₂₅H₂₆N₆O requires 426.51).

### 4-(1H-indol-3-yl)-N-(3-methyl-4-thiomorpholin-4-ylphenyl)pyrimidin-2-amine (28)

Anal, RP-HPLC: t_{R} 16.86 min. (10-70% MeCN).

¹H NMR (DMSO-d₆, 500 MHz): δ_{H} 2.26 (3H, s, CH₃), 2.76 (4H, dd, J 5.0, 2×CH₂), 3.06 (4H, dd, J 5.0, 2×CH₂), 7.00 (1H, d, J 8.5, Ar—H), 7.12 (1H, dd, J 7.5, Ar—H), 7.20 (1H, dd, J 7.5, Ar—H), 7.22 (1H, d, J 5.0, pyrim-H), 7.46 (1H, d, J 7.5, Ar—H), 7.50 (1H, dd, J 8.5, 2.0, Ar—H), 7.72 (1H, d, J 2.0, Ar—H), 8.28 (2H, m, incl. J 5.0, pyrim-H and Ar—H), 8.59 (1H, d, J 7.5, Ar—H), 9.18 (1H, s, Ar—H) and 11.76 (1H, s, NH).

MS (ESI⁺) m/z 402.31 [M+H]⁺ (C₂₃H₂₃N₅S requires 401.53).

### 4-(1H-indol-3-yl)-N-[(2R,6S)-2,6-dimethylmorpholin-4-ylphenyl]pyrimidin-2-amine (29)

The cis isomer was prepared from guanidine synthesised using cis-2,6-dimethylmorpholine. Characterisation data for Compound (29) was essentially identical to Compound (9) (Compound (9) contains ˜5% of Compound (30) below)

### 4-(1H-indol-3-yl)-N-[(2S,6S)-2,6-dimethylmorpholin-4-ylphenyl]pyrimidin-2-amine (30)

The trans isomer was obtained from Prep-HPLC (0.1% TFA) on batch 02 of Compound (9).

Anal. RP-HPLC: t_{R} 14.76 min. (10-70% MeCN).

¹H NMR (DMSO-d₆, 500 MHz): δ_{H} 1.27 (6H, d, J 6.5, 2×CH₃), 2.81-2.86 (2H, m, 2×CHH), 3.16-3.21 (2H, m, 2×CHH), 4.06-4.10 (2H, m, 2×CHCH₃), 6.98 (2H, dd, J 6.5, 2×Ar—H), 7.13 (1H, dd, J 7.5, Ar—H), 7.21 (1H, dd, J 7.5, Ar—H), 7.30 (1H, br s, pyrim-H), 7.52-7.56 (2H, m, 2×Ar—H), 7.48 (1H, d, J 7.5, Ar—H), 8.19 (1H, br s, pyrim-H), 8.46 (2H, dd, J 6.5, 2×Ar—H), 9.65 (1H, br s, NH) and 12.03 (1H, s, NH).

MS (ESI⁺) m/z 400.35 [M+H]⁺ (C₂₄H₂₅N₅O requires 399.49).

### 4-(1H-indol-3-yl)-N-(3,5-dimethylpiperidin-1-ylphenyl)pyrimidin-2-amine (31)

The 4:1 cis:trans mixture obtained was the result of a cyclisation with a guanidine containing a 4:1 cis:trans ratio of diastereoisomers.

Anal. RP-HPLC: t_{R} 11.95 min. (cis; 90%), 12.54 min. (trans; 10%) (10-70% MeCN).

¹H NMR (DMSO-d₆, 500 MHz): δ_{H} (cis) 0.91 (6H, d, J 6.5, 2×CH₃), 1.69-1.77 (2H, m, CH₂), 2.11 (2H, dd, J 11.5, 2×CHH), 3.55 (2H, d, J 11.5, 2×CHH), 3.56-4.17 (2H, m, 2×CHCH₃), 6.90 (2H, d, J 8.5, 2×Ar—H), 7.11 (1H, dd, J 8.5, 2×Ar—H), 7.17-7.20 (2H, m, incl. J 8.5, Ar—H and pyrim-H), 7.45 (1H, d, J 8.5, Ar—H), 7.61 (2H, d, J 8.5, 2×Ar—H), 7.66-7.73 (2H, m, 2×Ar—H), 8.26 (2H, d, incl. J 5.5, pyrim-H and Ar—H), 8.57 (1H, d, J 7.5, Ar—H), 8.46 (2H, dd, J 6.5, 2×Ar—H), 9.07 (1H, s, NH) and 11.73 (1H, 9, NH); δ_{H} (trans-observable signals) 1.00 (6H, d, J 6.5, 2×CH₃), 1.60-1.64 (2H, m, CH₂), 1.95-2.05 (2H, m, 2×CHH), 2.72 (2H, dd, J 11.5, 6.5, 2×CHH), 3.08 (2H, dd, J 11.5, 4.0, 2×CHH).

MS (ESI⁺) m/z 398.26 [M+H]⁺ (C₂₅H₂₇N₅ requires 397.52).

### Example 6

### Kinase Assays

The compounds from the examples above were investigated for their ability to inhibit the enzymatic activity of various protein kinases. This was achieved by measurement of incorporation of radioactive phosphate from ATP into appropriate polypeptide substrates. Recombinant protein kinases and kinase complexes were produced or obtained commercially. Assays were performed using 96-well plates and appropriate assay buffers (typically 25 mM β-glycerophosphate, 20 mM MOPS, 5 mM EGTA, 1 mM DTT, 1 mM Na₃VO₃, pH 7.4), into which were added 2-4 μg of active enzyme with appropriate substrates. The reactions were initiated by addition of Mg/ATP mix (15 mM MgCl₂+100 μM ATP with 30-50 kBq per well of [γ-³²P]-ATP) and mixtures incubated as required at 30° C. Reactions were stopped on ice, followed by filtration through p81 filterplates or GF/C filterplates (Whatman Polyfiltronics, Kent, UK). After washing 3 times with 75 mM aq orthophosphoric acid, plates were dried, scintillant added and incorporated radioactivity measured in a scintillation counter (TopCount, Packard Instruments, Pangbourne, Berks, UK). Compounds for kinase assay were made up as 10 mM stocks in DMSO and diluted into 10% DMSO in assay buffer. Data was analysed using curve-fitting software (GraphPad Prism version 3.00 for Windows, GraphPad Software, San Diego Calif. USA) to determine IC₅₀ values (concentration of test compound which inhibits kinase activity by 50%). Results for representative example compounds are summarised in Tables 2 and 3.

### CDK 7 and 9 Assays

CTD peptide substrate (biotinyl-Ahx-(Tyr-Ser-Pro-Thr-Ser-Pro-Ser)₄-NH₂; 1-2 mg/mL) and recombinant human CDK7/cyclin H, CDK9/cyclin TI, or CDK9/cyclin K (0.5-2 μg) were incubated for 45 min at 30° C. in the presence of varying amounts of test compound in 20 mM MOPS pH 7.2, 25 mM β-glycerophosphate, 5 mM EGTA, 1 mM DTT, 1 mM sodium vanadate, 15 mM MgCl₂, and 100 μM ATP (containing a trace amount of ³²PγATP) in a total volume of 25 μL in a 96-well microtiter plate. The reaction was stopped by placing the plate on ice for 2 min. Avidin (50 μg) was added to each well, and the plate was incubated at room temp for 30 min. The samples were transferred to a 96-well P81 filter plate, and washed (4×200 μL per well) with 75 mM phosphoric acid. Microscint 40 scintillation liquid (50 μL) was added to each well, and the amount of ³²P incorporation for each sample was measured using a Packard Topcount microplate scintillation counter. Results for representative example compounds are summarised in Tables 2 and 3.

### Aurora-A (Human) Kinase Assay

This was achieved by measurement of incorporation of radioactive phosphate from ATP into Kemptide substrate (LRRASLG), upon phosphorylation by commercially obtained aurora-A (human, Upstate, Dundee, UK). Assays were performed using 96-well plates and appropriate assay buffers (20 mM Tris, 25 mM β-glycerophosphate, 5 mM EGTA, 1 mM DTT, 1 mM sodium vanadate, pH 7.5), into which were added 2-5 ng of active enzyme with 500 μM substrate (Kemptide). The reactions were initiated by addition of MgATP mix (15 mM MgCl₂+100 μM ATP with 15-25 kBq per well of [γ-³²P]-ATP) and mixtures incubated for 30 min at 30° C. Reactions were stopped by addition of an equal volume of 75 mM aq orthophosphoric acid, followed by filtration through p81 filterplates (Whatman Polyfiltronics, Kent, UK). After washing 4 times with 75 mM aq orthophosphoric acid, plates were dried, scintillant added and incorporated radioactivity measured in a scintillation counter (TopCount, Packard Instruments, Pangbourne, Berks, UK). Compounds for kinase assay were made up as 10 mM stocks in DMSO and diluted into 10% DMSO in assay buffer. Data was analysed using curve-fitting software (XLfit version 4.0.2, IDBS, Guildford, Surrey, UK) to determine IC₅₀ values (concentration of test compound which inhibits kinase activity by 50%).

### Aurora-B (Human) Kinase Assay

This was achieved by measurement of incorporation of radioactive phosphate from ATP into Kemptide substrate (LRRASLG), upon phosphorylation by commercially obtained aurora-B (human, Upstate, Dundee, UK). Assays were performed using 96-well plates and appropriate assay buffers (20 mM Tris, 25 mM β-glycerophosphate, 5 mM EGTA, 1 mM DTT, 1 mM sodium vanadate, pH 7.5), into which were added 75 ng of pre-activated enzyme with 500 μM substrate (Kemptide). The reactions were initiated by addition of MgATP mix (15 mM MgCl₂+100 μM ATP with 15-25 kBq per well of [γ-³²P]-ATP) and mixtures incubated for 60 min at 30° C. Reactions were stopped by addition of an equal volume of 75 mM aq orthophosphoric acid, followed by filtration through p81 filterplates (Whatman Polyfiltronics, Kent, UK). After washing 4 times with 75 mM aq orthophosphoric acid, plates were dried, scintillant added and incorporated radioactivity measured in a scintillation counter (TopCount, Packard Instruments, Pangbourne, Berks, UK). Compounds for kinase assay were made up as 10 mM stocks in DMSO and diluted into 10% DMSO in assay buffer. Data was analysed using curve-fitting software (XLfit version 4.0.2, IDBS, Guildford, Surrey, UK) to determine IC₅₀ values (concentration of test compound which inhibits kinase activity by 50%).

### Pre-Activation of Aurora-B (Human)

Aurora-B (human, Upstate, Dundee, UK) was pre-activated immediately prior to kinase assay in appropriate buffers (20 mM Tris, 25 mM β-glycerophosphate, 5 mM EGTA, 1 mM DTT, 1 mM sodium vanadate, pH 7.5) by incubating 15 μg of enzyme with 4 μg INCENP (Upstate, Dundee, UK) in the presence of MgATP mix (15 mM MgCl₂+100 μM ATP) for 15 min at 30° C.

### Flt3 Kinase Assay

This was achieved by measurement of incorporation of radioactive phosphate from ATP into myelin basic protein (MBP) substrate, upon phosphorylation by commercially obtained Flt-3 Upstate, Dundee, UK). Assays were performed using 96-well plates and appropriate assay buffers (20 mM Tris, 25 mM β-glycerophosphate, 5 mM EGTA, 1 mM DTT, 1 mM sodium vanadate, pH 7.5), into which were added 5 ng of active enzyme with 0.4 mg/ml substrate (MBP). The reactions were initiated by addition of MgATP mix (15 mM MgCl₂+100 μM ATP with 15-25 kBq per well of [γ-³²P]-ATP) and mixtures incubated for 30 min at 30° C. Reactions were stopped by addition of an equal volume of 75 mM aq orthophosphoric acid, followed by filtration through p81 filterplates (Whatman Polyfiltronics, Kent, UK). After washing 4 times with 75 mM aq orthophosphoric acid, plates were dried, scintillant added and incorporated radioactivity measured in a scintillation counter (TopCount, Packard Instruments, Pangbourne, Berks, UK). Compounds for kinase assay were made up as 10 mM stocks in DMSO and diluted into 10% DMSO in assay buffer. Data was analysed using curve-fitting software (XLfit version 4.0.2, IDBS, Guildford, Surrey, UK) to determine IC₅₀ values (concentration of test compound which inhibits kinase activity by 50%).

### GSK-3β Kinase Assay

GSK-3 was obtained from New England Biolabs (UK) Ltd., Hitchin, Herts. The recombinant enzyme was isolated from a strain of E. coli that carries a clone expressing GSK-3β derived from a rabbit skeletal muscle cDNA library [Wang, Q. M.; Fiol, C. J.; DePaoli-Roach, A. A.; Roach, P. J. J. Biol. Chem., 1994, 269, 14566]. Inhibition of GSK-3 function was assessed by measurement of phosphorylation of CREB phosphopeptide KRREILSRRPphosphoSYR in the presence of test compounds. Using a 96-well assay format, GSK3 (7.5 U) was incubated for 30 min at 30° C. in a total volume of 25 μL in 20 mM MOPS pH 7.2, 25 mM β-glycerophosphate, 5 mM EGTA, 1 mM DTT, 1 mM Na₃VO₃, 40 μM CREB peptide, 15 mM MgCl₂ and 100 μM ATP (containing 0.25 μCi [γ-³²P]-ATP) in the presence of varying concentrations of test compound. The samples were transferred to 96-well p81 filter plates (Whatman Polyfiltronics, Kent, UK), and the plates were washed 4 times with 200 μL/well of 75 mM aq orthophosphoric acid. Scintillation liquid (50 μL) was added to each well, and incorporated radioactivity for each sample was determined using a scintillation counter (TopCount, Packard Instruments, Pangbourne, Berks, UK). Results for representative example compounds are summarised in Tables 2 and 3.

### Example 7

### MTT Cytotoxicity Assay

The compounds of the invention were subjected to a standard cellular proliferation assay using human tumour cell lines obtained from the ATCC (American Type Culture Collection, 10801 University Boulevard, Manessas, Va. 20110-2209, USA). Standard 72-h MTT (thiazolyl blue; 3-[4,5-dimethylthiazol-2-yl]-2,5-diphenyltetrazolium bromide) assays were performed (Haselsberger, K.; Peterson, D. C.; Thomas, D. G.; Darling, J. L. Anti Cancer Drugs 1996, 7, 331-8; Loveland, B. E.; Johns, T. G.; Mackay, 1. R.; Vaillant, F.; Wang, Z. X.; Hertzog, P. J. Biochemistry International 1992, 27, 501-10). In short: cells were seeded into 96-well plates according to doubling time and incubated overnight at 37° C. Test compounds were made up in DMSO and a 1/3 dilution series prepared in 100 μL cell media, added to cells (in triplicates) and incubated for 72 ho at 37° C. MTT was made up as a stock of 5 mg/mL in cell media and filter-sterilised. Media was removed from cells followed by a wash with 200 μL PBS. MTT solution was then added at 20 μL per well and incubated in the dark at 37° C. for 4 h MTT solution was removed and cells again washed with 200 μL PBS. MTT dye was solubilised with 200 μL per well of DMSO with agitation. Absorbance was read at 540 nm and data analysed using curve-fitting software (GraphPad Prism version 3.00 for Windows, GrapbPad Software, San Diego Calif. USA) to determine IC₅₀ values (concentration of test compound which inhibits cell growth by 50%). Results for representative example compounds are summarised in Table 4.

Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the relevant fields are intended to be within the scope of the following claims.

TABLE 1
Structures of exemplified compounds

| Compound | | |
|---|---|---|
| No. | Name | Structure |
| 1 | 4-(1H-indol-3-yl)pyrimidin-2- amine | 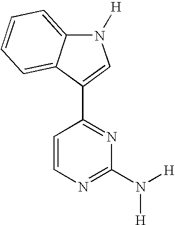 |
| 2 | 4-(1H-indol-3-yl)-N-(3- nitrophenyl)pyrimidin-2-amine | 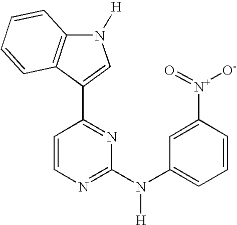 |
| 3 | N-(4-fluorophenyl)-4-(1H-indol- 3-yl)pyrimidin-2-amine | 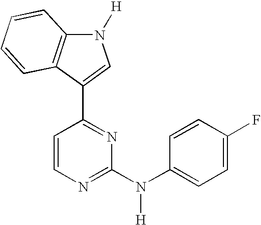 |
| 4 | 4-(1H-indol-3-yl)-N-(6- methoxypyridin-3-yl)pyrimidin-2- amine | 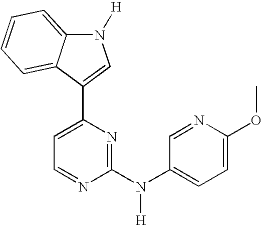 |
| 5 | 4-(1H-indol-3-yl)-N-(4- morpholin-4-ylphenyl)pyrimidin- 2-amine | 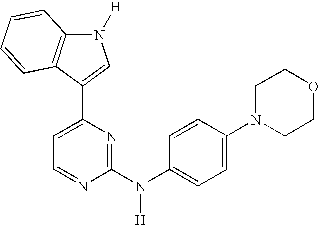 |
| 6 | 4-(1H-indol-3-yl)-N-(4- acetylpiperazin-1- ylphenyl)pyrimidin-2-amine | 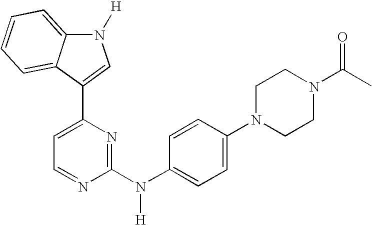 |
| 7 | 4-(1H-indol-3-yl)-N-(4-piperazin- 1-ylphenyl)pyrimidin-2-amine | 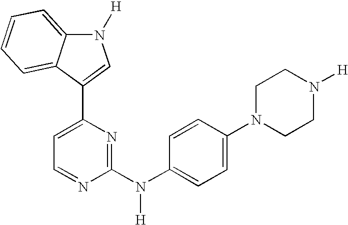 |
| 8 | 4-(1H-indol-3-yl)-N-(4- benzylpiperazin-1- ylphenyl)pyrimidin-2-amine | 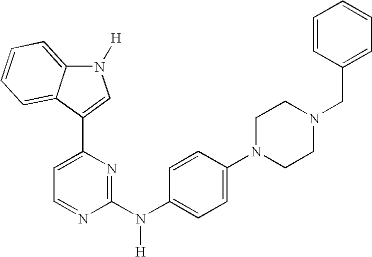 |
| 9 | 4-(1H-indol-3-yl)-N-(2,6- dimethylmorpholin-4- ylphenyl)pyrimidin-2-amine | 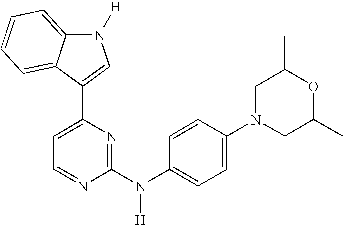 |
| 10 | N′-[4-(1H-indol-3-yl)pyrimidin-2- yl]-N,N-dimethylbenzene-1,4- diamine | 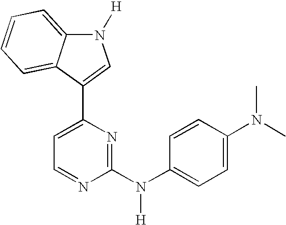 |
| 11 | 4-(1H-indol-3-yl)-N-(2-methyl-4- morpholin-4-ylphenyl)pyrimidin- 2-amine | 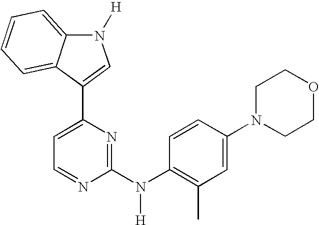 |
| 12 | 4-(1H-indol-3-yl)-N-(3,4,5- trimethoxyphenyl)pyrimidin-2- amine | 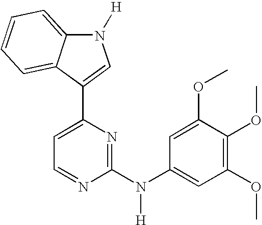 |
| 13 | 4-(1H-indol-3-yl)-N-(3-methoxyl- 4-morpholin-4- ylphenyl)pyrimidin-2-amine | 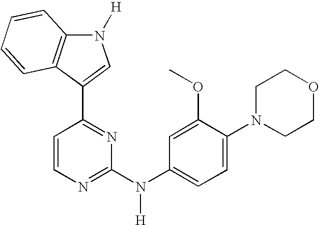 |
| 14 | N-(3,5-dimethoxyphenyl)-4-(1H- indol-3-yl)pyrimidin-2-amine | 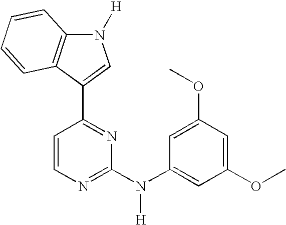 |
| 15 | 4-(1-methyl-1H-indol-3-yl)-N-(4- morpholin-4-ylphenyl)pyrimidin- 2-amine | 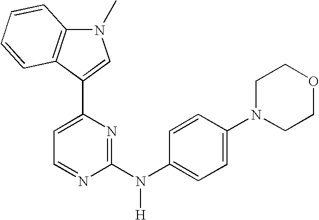 |
| 16 | 4-(1-methyl-1H-indol-3-yl)-N-(4- acetylpiperazine-1- ylphenyl)pyrimidin-2-amine | 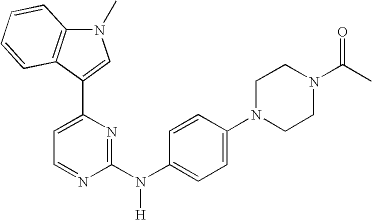 |
| 17 | N-1,3-benzodioxol-5-yl-4-(1H- indol-3-yl)pyrimidin-2-amine | 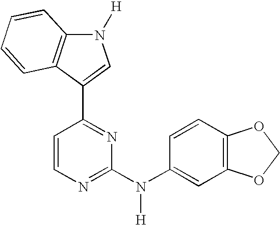 |
| 18 | 4-[1-(cyclopropylcarbonyl)-1H- indol-3-yl]-N-(4-morpholin- 4ylphenyl)pyrimidin-2-amine | 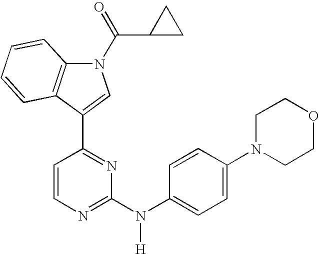 |
| 19 | 4-(1-acetyl-1H-indol-3-yl)-N-(4- morpholin-4ylphenyl)pyrimidin- 2-amine | 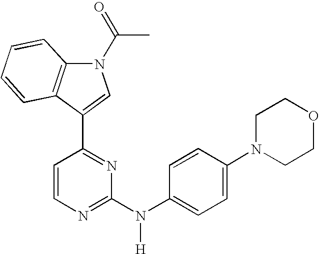 |
| 20 | 4-(1H-indol-3-yl)-N-(4- methylpiperazin-1- ylphenyl)pyrimidin-2-amine | 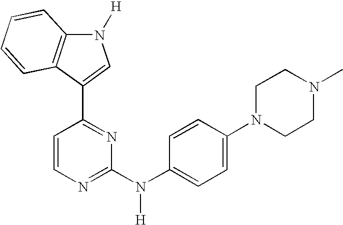 |
| 21 | 4-(7-methoxy-1H-indol-3-yl)-N- (4-acetylpiperazin-1- ylphenyl)pyrimidin-2-amine | 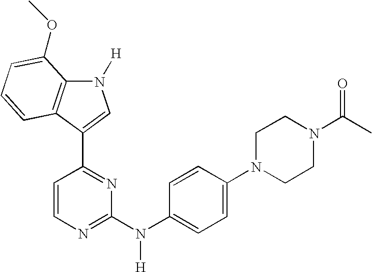 |
| 22 | 4-(2-methyl-1H-indol-3-yl)-N-(4- acetylpiperazin-1- ylphenyl)]pyrimidin-2-amine | 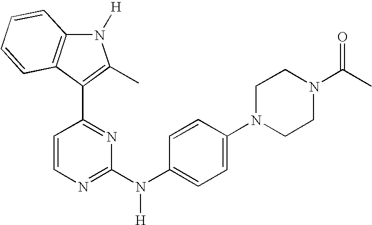 |
| 23 | 4-(7-methyl-1H-indol-3-yl)-N-(4- acetylpiperazin-1- ylphenyl)]pyrimidin-2-amine | 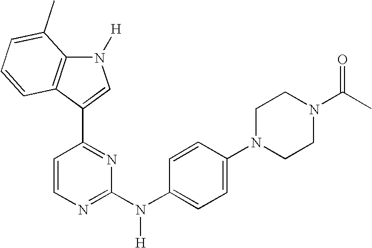 |
| 24 | 4-(6-methoxy-1H-indol-3-yl)-N- (4-acetylpiperazin-1- ylphenyl)]pyrimidin-2-amine | 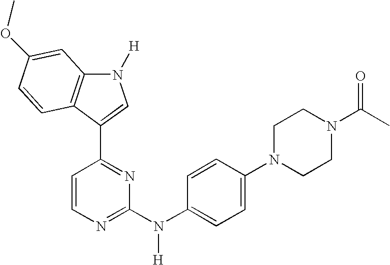 |
| 25 | 4-(7-chloro-1H-indol-3-yl)-N-(4- acetylpiperazin-1- ylphenyl)]pyrimidin-2-amine | 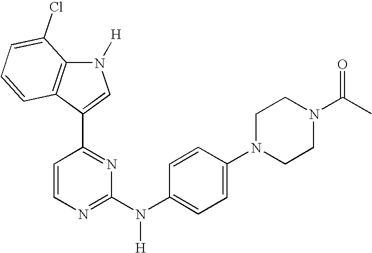 |
| 26 | 4-(6-fluoro-1H-indol-3-yl)-N-(4- acetylpiperazin-1- ylphenyl)]pyrimidin-2-amine | 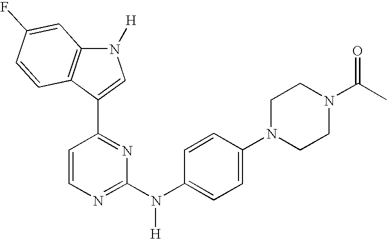 |
| 27 | 4-(1H-indol-3-yl)-N-[(4- acetylpiperazin-1-yl)-3- methylphenyl]pyrimidin-2-amine | 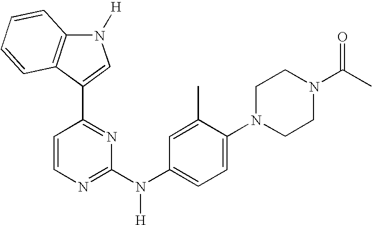 |
| 28 | 4-(1H-indol-3-yl)-N-(3-methyl-4- thiomorpholin-4- ylphenyl)pyrimidin-2-amine | 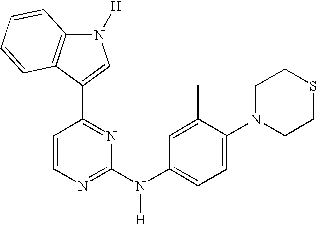 |
| 29 | 4-(1H-indol-3-yl)-N-[(2R,6S)-2,6- dimethylmorpholin-4- ylphenyl]pyrimidin-2-amine | 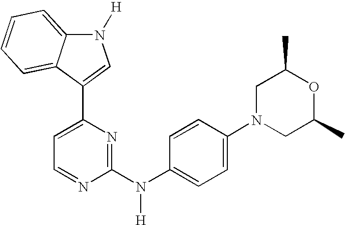 |
| 30 | 4-(1H-indol-3-yl)-N-[(2S,6S)-2,6- dimethylmorpholin-4- ylphenyl]pyrimidin-2-amine | 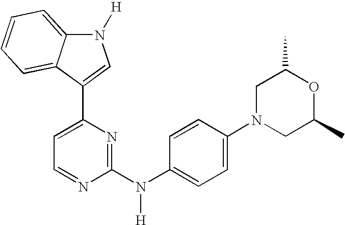 |
| 31 | 4-(1H-indol-3-yl)-N-(3,5- dimethylpiperidin-1- ylphenyl)pyrimidin-2-amine | 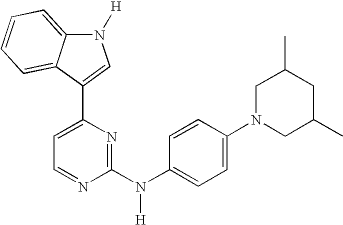 |

TABLE 2
Kinase inhibition of selected compounds

| | Kinase inhibition IC₅₀ (μM) | | | | | | | | |
|---|---|---|---|---|---|---|---|---|---|
| Compound | Aurora A | FLT3 | CDK1B | CDK2A | CDK2E | CDK4D1 | CDK7H | CDK9T1 | GSK3 |
| 2 | 0.27 | 0.13 | >100 | >100 | 2.9 | >100 | >100 | 6.1 | 2.1 |
| 3 | 1.1 | 0.27 | >100 | >100 | 1.2 | >100 | >100 | >100 | 8.7 |
| 4 | 0.24 | 0.15 | 1.9 | 2.6 | 1.3 | 1.0 | 5.4 | 2.8 | 1.6 |
| 5 | 0.058 | 0.052 | | | >10 | | >10 | >10 | |

TABLE 3
Kinase inhibition of selected compounds

| Compound | CDK2E | CDK4D1 | CDK7H | CDK9T1 | Aurora A | Aurora B | Flt3 |
|---|---|---|---|---|---|---|---|
| 2 | 2.9473 | | | 6.0755 | 0.2738* | | 0.1332 |
| 3 | 1.1707 | | | | 1.0516* | | 0.2742 |
| 4 | 1.2531 | 1.0025 | 5.4430 | 2.8170 | 0.2434* | | 0.1481 |
| 5 | | | | | 0.8044 | 0.2596 | 0.0516 |
| 6 | 4.5026 | 4.3941 | | 2.0838 | 0.1297 | 0.1254 | 0.0133 |
| 7 | 1.9309 | 0.4261 | 2.5284 | 0.5200 | 0.2210 | 0.1702 | 0.0127 |
| 8 | | | 6.0420 | | | 1.4056 | 0.0788 |
| 9 | 4.9098 | 5.4568 | 6.6715 | 3.0325 | 0.2766 | 0.1582 | 0.0298 |
| 10 | 2.3539 | 5.7774 | 5.2264 | 1.5950 | 0.2820 | 0.7288 | 0.0849 |
| 11 | 0.3318 | | | | | | 0.7258 |
| 12 | 1.8163 | | | 1.2560 | 0.0534 | 0.0664 | 0.0013 |
| 13 | 7.5972 | | | 4.2449 | 0.1117 | 0.2280 | 0.0024 |
| 14 | | | | | 0.5632 | | |
| 15 | | | | | 0.3695 | | |
| 16 | | | | | 0.2345 | | |
| 17 | | | | | 0.7419 | | |
| 18 | | | | | 3.3642 | | |
| 19 | | | | | 2.1875 | | |
| 20 | | | | | 0.4340 | | |
| 21 | | | | | 0.2339 | | |
| 22 | 5.1530 | | 6.6620 | 4.4351 | 0.0377 | 0.0246 | 0.0110 |
| 23 | 2.4298 | | 3.4710 | 1.5390 | 0.0917 | 0.0761 | 0.0332 |
| 24 | | | | | 0.2105 | | |
| 25 | | | | | 0.5592 | | |
| 26 | | | | | 0.1938 | 0.1747 | |
| 27 | | | | | 0.2761 | 0.3652 | |
| 28 | | | | | 0.8560 | 0.6364 | |
| 29 | | | | | 0.3858 | | |
| 30 | | | | | 1.2479 | | |
| 31 | | | | | 4.5139 | | |

*Drosophila Aurora A

TABLE 4
MTT Cytoxicity data for cell lines Mia-PaCa-2 and A2780
(IC₅₀ values in μM after 96 hours incubation)
for selected compounds of the invention

| Compound | Mia-PaCa-2 | A2780 |
|---|---|---|
| 5 | 2.404 | 1.032 |
| 6 | 0.785 | 0.789 |
| 7 | 0.166 | 0.086 |
| 8 | 5.455 | 5.426 |
| 9 | 6.232 | 5.446 |
| 10 | 5.478 | 0.485 |
| 11 | 6.822 | 4.660 |
| 12 | 0.077 | 0.043 |
| 13 | 6.729 | 2.485 |
| 21 | 1.036 | |
| 22 | 0.778 | 0.299 |
| 23 | 0.789 | 0.448 |
| 24 | 0.723 | 0.576 |

### REFERENCES

1. Manning, G.; Whyte, D. B.; Martinez, R.; Hunter, T.; Sudarsanam, S. The protein kinase complement of the human genome. Science 2002, 298, 1912-1934.
2. Kostich, M.; English, J.; Madison, V.; Gheyas, F.; Wang, L. et al. Human members of the eukaryotic protein kinase family. Genome Biology 2002, 3, research 0043.0041-0043.0012.
3. Dancey, J.; Sausville, E. A. Issues and progress with protein kinase inhibitors for cancer treatment. Nat. Rev. Drug Disc. 2003, 2, 296-313.
4. Cockerill, G. S.; Lackey, K. E. Small molecule inhibitors of the class 1 receptor tyrosine kinase family. Current Topics in Medicinal Chemistry 2002, 2, 1001-1010.
5. Fabbro, D.; Ruetz, S.; Buchdunger, E.; Cowan-Jacob, S. W.; Fendrich, G. et al. Protein kinases as targets for anticancer agents: from inhibitors to useful drugs. Pharmacol. Ther. 2002, 93, 79-98.
6. Cohen, P. Protein kinases—the major drug targets of the twenty-first century? Nat. Rev. Drug Disc. 2002, 1, 309-315.
7. Bridges, A. J. Chemical inhibitors of protein kinases. Chem. Rev: 2001, 101(8), 2541-2571.
8. Wang, S.; Meades, C.; Wood, G.; Osnowski, A.; Fischer, P. M. N-(4-(4-methylthiazol-5-yl)pyrimidin-2-yl)-N-phenylamines as antiproliferative compounds. PCT Intl. Patent Appl. Publ. WO 2003029248; Cyclacel Limited, UK.
9. Wu, S. Y.; McNae, I.; Kontopidis, G.; McClue, S. J.; McInnes, C. et al. Discovery of a Novel Family of CDK Inhibitors with the Program LIDAEUS: Structural Basis for Ligand-Induced Disordering of the Activation Loop. Structure 2003, 11, 399-410.
10. Fischer, P. M.; Wang, S.; Wood, G. Inhibitors of cyclin dependent kinases as anti-cancer agent. PCT Intl. Patent Appl. Publ. WO 02/079193; Cyclacel Limited, UK.
11. Wang, S.; Fischer, P. M. Anti-cancer compounds. US Patent Appl. Publ. 2002/0019404.
12. Fischer, P. M.; Wang, S. 2-substituted 4-heteroaryl-pyrimidines and their use in the treatment of proliferative disorders. PCT Intl. Patent Appl. Publ. WO 2001072745; Cyclacel Limited, UK.
13. Knockaert, M.; Greengard, P.; Meijer, L. Pharmacological inhibitors of cyclin-dependent kinases. Trends Pharmacol. Sci. 2002, 23, 417-425.
14. Fischer, P. M.; Endicott, J.; Meijer, L. Cyclin-dependent kinase inhibitors. Progress in Cell Cycle Research; Editions de la Station Biologique de Roscoff: Roscoff, France, 2003; pp 235-248.
15. Fravolini, A.; Grandolini, G.; Martani, A. New heterocyclic ring systems from α-hydroxymethylene ketones. V. Reaction of 2-methyl-6-hydroxymethylene-4,5,6,7-tetrahydrobenzothiazol-7-one with amines and amidines. Gazz. Chim. Ital. 1973, 103, 1063-1071.
16. Cleaver, L.; Croft, J. A.; Ritchie, E.; Taylor, W. C. Chemical studies of the Proteaceae. IX. Synthesis of 5-alkylresorcinols from aliphatic precursors. Aust. J. Chem. 1976, 29, 1989-2001.
17. Fadda, A. A.; El-Houssini, M. S. Synthesis of cyclic ketones by activated nitriles. J. Ind. Chem. Soc. 1990, 67, 915-917.
18. Kost, A. N.; Ovseneva, L. G. Synthesis of 4-substituted dihydroresorcinols. Zh. Obshch. Khim 1962, 32, 3983-3986.
19. Lehmann, G.; Luecke, B.; Schick, H.; Hilgetag, G. 2-Substituted 7-oxo-4,5,6,7-tetrahydrobenzothiazoles. Z. Chem. 1967, 7, 422.
20. Bell, R. P.; Davis, G. G. Kinetics of the bromination of some enols and their anions. J. Chem. Soc 1965, 353-361.
21. Fravolini, A.; Grandolini, G.; Martani, A. New heterocyclic ring systems from α-hydroxymethylene ketones. III. Pyrazolobenzothiazoles and thiazolo-benzoisoxazoles. Gazz. Chim. Ital. 1973, 103, 755-769.
22. Bredereck, H.; Effenberger, F.; Botsch, H. Acid amide reactions. XLV. Reactivity of formamidines, dimethylformamide diethyl acetal (amide acetal), and bis(dimethylamino)methoxymethane (aminal ester). Chem. Ber. 1964, 97, 3397-3406.
23. Wang D, De la Fuente C, Deng L, Wang L, Zilberman I, Eadie C, Healey M, Stein D, Denny T, Harrison L E, Meijer L, Kashanchi F. Inhibition of human immunodeficiency virus type 1 transcription by chemical cyclin-dependent kinase inhibitors. J. Virol. 2001; 75: 7266-7279.
24. Chen, Y. H.; Hansen, L.; Chen, M. X.; Bjorbaek, C.; Vestergaard, H.; Hansen, T.; Cohen, P. T.; Pedersen, O. Diabetes, 1994, 43, 1234.
25. Nikoulina, S. E.; Ciaraldi, T. P.; Mudaliar, S.; Mohideen, P.; Carter, L.; Henry, R. R. Diabetes, 2000, 49, 263.
26. Goedert, M. Curr. Opin. Gen. Dev., 2001, 11, 343.
27. Mattson, M. P. Nat. Rev. Mol. Cell. Biol., 2000, 1, 120.
28. Zhu, A. J.; Watt, F. M. Development, 1999, 126, 2285.
29. DasGupta, R.; Fuchs, E. Development, 1999, 126, 4557.
30. Sunkel et al., J. Cell Sci., 1988, 89, 25.
31. Llamazares et al., Genes Dev., 1991, 5, 2153.
32. Glover et al., Genes Dev., 1998, 12, 3777.
33. Lee et al., Proc. Natl. Acad. Sci. USA, 1998, 95, 9301.
34. Leung et al., Nat. Struct. Biol., 2002, 9, 719.
35. Kauselmann et al., EMBO J., 1999, 18, 5528.
36. Nigg, Curr. Opin Cell Biol., 1998, 10, 776.
37. Yuan et al., Cancer Res., 2002, 62, 4186.
38. Seong et al., J. Biol. Chem., 2002, 277, 32282.
39. Lane et al., J. Cell. Biol., 1996, 135, 1701.
40. Cogswell et al., Cell Growth Differ., 2000, 11, 615.
41. Liu et al., Proc. Natl. Acad. Sci. USA, 2002, 99, 8672.
42. Toyoshima-Morimoto et al., Nature, 2001, 410, 215.
43. Roshak et al., Cell. Signalling, 2000, 12, 405.
44. Smits et al., Nat. Cell Biol., 2000, 2, 672.
45. van Vugt et al., J. Biol. Chem., 2001, 276, 41656.
46. Sumara et al., Mol. Cell, 2002, 9, 515.
47. Golan et al., J. Biol. Chem., 2002, 277, 15552.
48. Kotani et al., Mol. Cell, 1998, 1, 371.
49. Feng et al., Cell Growth Differ., 2001, 12, 29.
50. Dai et al., Oncogene, 2002, 21, 6195.
51. Nurse, Nature, 1990, 344, 503.
52. Nigg, Nat. Rev. Mol. Cell. Biol., 2001, 2, 21.
53. Hagting et al., EMBO J., 1998, 17, 4127.
54. Hagting et al., Curr. Biol., 1999, 9, 680.
55. Yang et al., J. Biol. Chem., 2001, 276, 3604.
56. Takizawa et al., Curr. Opin. Cell Biol., 2000, 12, 658.
57. Seki et al., Mol. Biol. Cell, 1992, 3, 1373.
58. Heald et al., Cell, 1993, 74, 463.
59. Dalal et al., Mol. Cell. Biol., 1999, 19, 4465.
60. Toyoshima-Morimoto et al., Nature, 2001, 410, 215.
61. Toyoshima-Morimoto et al., EMBO Rep., 2002, 3, 341.
62. Wang et al., Mol. Cell. Biol., 2002, 22, 3450.
63. Tyrrell, E.; Brookes, P. Synthesis 2003, 469-483.
64. Molander, G. A.; Biolatto, B. J. Org. Chem. 2003, 68, 43024314.
65. Bredereck, H.; Effenberger, F.; Botsch, H. Chem. Ber. 1964, 97, 3397-3406.
66. Zimmermann, J.; Caravatti, G.; Mett, H.; Meyer, T.; Müller, M. et al. Arch. Pharm. Pharm. Med. Chem. 1996, 329, 371-376.
67. Haselsberger, K.; Peterson, D. C.; Thomas, D. G.; Darling, J. L. Anti Cancer Drugs 1996, 7, 331-8.
68. Loveland, B. E.; Johns, T. G.; Mackay, I. R.; Vaillant, F.; Wang, Z. X.; Hertzog, P. J. Biochemistry International 1992, 27, 501-10.
69. Joule, J. A.; Smith, G. F. Heterocyclic Chemistry, Van Nostrand Reinhold (UK) Co. Ltd.: Wokingham, 1983.
70. Bredereck, H.; Effenberger, F.; Botsch, H. Chem. Ber., 1964, 97, 3397.
71. Cohen, P. Nat. Rev. Drug Disc., 2002, 1, 309.
72. Fischer, P. M. Curr. Med. Chem., 2004, 11, 1563.
73. Kidwai, M.; Rastogi, S.; Saxena, S. Bulletin of the Korean Chemical Society, 2003, 24, 1575.
74. Zimmermann, J.; Caravatti, G.; Mett, H.; Meyer, T.; Mueller, M.; Lydon, N. B.; Fabbro, D. Arch. Pharm. (Weinheim), 1996, 329, 371.
75. Carmena, M.; Earnshaw, W. C. Nat. Rev. Mol. Cell Biol., 2003, 4, 842.
76. Stirewalt, D. L.; Radich, J. P. Nat. Rev. Cancer, 2003, 3, 650.
77. Fischer, P. M.; Endicott, J.; Meijer, L. Progr. Cell Cycle Res., 2003, 5, 235.
78. Cohen, P.; Goedert, M. Nat. Rev. Drug Disc., 2004, 3, 479.
79. Reilly, J. T. Leukemia & Lymphoma, 2003, 44, 1.

1. A compound of formula I, or a pharmaceutically acceptable salt thereof, 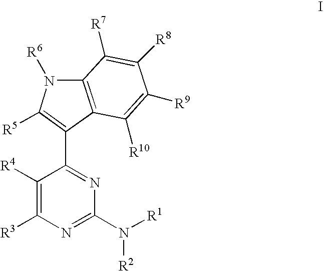
wherein R³, R⁴, R⁵, R⁶, R⁷, R⁸, R⁹, and R¹⁰ are each independently H, R¹¹ or R¹²;
R¹ and R² are each independently H. R¹¹ or R¹²; or R¹ and R² are linked to form a cyclic group together with the nitrogen to which they are attached, and wherein said cyclic group is optionally substituted with one or more R¹¹ or R¹² groups;
each R¹¹ is independently a hydrocarbyl group optionally substituted by one or more R¹² substituents;
each R¹² is independently selected from OR¹³, COR¹³, COOR¹³, CN, CONR¹³R¹⁴, NR¹³R¹⁴, SR¹³, SOR¹³, SO₂R¹³, SO₂OR¹³, SO₂NR¹³R¹⁴, R¹³, halogen, CF₃, NO₂ and an alicyclic group itself optionally substituted by one or more R¹² or R¹³ groups; and
each R¹³ and each R¹⁴ are independently H or (CH₂)ₙR⁵, where n is 0, 1, 2, or 3; and
each R¹⁵ is independently selected from alkyl, cycloalkyl, aryl, heteroaryl, aryl and an alicyclic group;
with the proviso that the compound is other than:
[4-(1H-indol-3-yl)-pyrimidin-2-yl]-[3-(1,1,2,2-tetrafluoroethoxyphenyl)]-amine;
3-[6-(4-bromophenyl)-2-(1-piperazinyl)-4-pyrimidinyl]-1H-indole;
3-[6-(4-bromophenyl)-2-(1-pyrrolidinyl)-4-pyrimidinyl]-1H-indole; or
3-[6-(4-bromophenyl)-2-(4-morpholinyl)-4-pyrimidinyl]-1H-indole.

2. A compound according to claim 1 wherein R¹ and R² are each independently H, R¹¹ or R¹²; or R¹ and R² are linked to form a cyclic group together with the nitrogen to which they are attached, wherein said cyclic group contains from two to nine carbon atoms and one or two heteroatoms selected from N, O, and S, and wherein said cyclic group is optionally substituted with one or two substituents selected from R¹¹ and R¹².

3. A compound according to claim 1 wherein R¹ and R² are each independently H, R¹¹ or R¹².

4. A compound according to claim 1 wherein R¹ and R² are each independently H or R¹¹.

5. A compound according to claim 1 wherein one of R¹ and R² is H and the other is R¹¹.

6. A compound according to claim 1 wherein R¹¹ is a hydrocarbyl group containing from 1 to 24 carbon atoms, optionally containing up to six heteroatoms selected from N, O, and S.

7. A compound according to claim 6 wherein the hydrocarbyl group is optionally substituted by up to six R¹² substituents.

8. A compound according to claims 1 wherein R¹¹ is an aryl group, a heteroaryl group, an aryl-alicyclic group or an alicyclic group, each of which may be optionally substituted by one or more R¹² substituents.

9. A compound according to claim 1 wherein R¹¹ is selected from phenyl, pyridinyl and 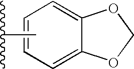
each of which may be optionally substituted by one or more R¹² substituents.

10. A compound according to claim 1 wherein R¹¹ is a phenyl or pyridinyl group, each of which may be optionally substituted by one or more R¹² substituents.

11. A compound according to claim 1 wherein R³, R⁴, R⁵, R⁶, R⁷, R⁸, R⁹, and R¹⁰ are each independently H or R¹².

12. A compound according to claim 1 wherein R³ and R⁴ are both H.

13. A compound according to claim 1 wherein:
R⁹, and R¹⁰ are both H;
R⁵ is H or alkyl;
R⁶ is H, alkyl, CO-alkyl or CO-cycloalkyl;
R⁷ is H, alkyl, alkoxy or halo; and
R⁸ is H, alkoxy or halo.

14. A compound according to claim 1 wherein each R¹⁵ is independently selected from ethyl, ethyl, isopropyl, n-butyl, isobutyl, t-butyl, cyclopropyl, cyclobutyl, cyclopentyl, cyclohexyl, phenyl, pyridinyl, pyrrolidinyl, pyrrolyl, morpholinyl, piperazinyl, piperidinyl, triazolyl, tetrazolyl and thiazolyl.

15. A compound according to claim 1 wherein the alicyclic group contains one or more heteroatoms.

16. A compound according to claim 1 wherein R¹² is an alicyclic group optionally substituted by one or more R¹³ or COR¹³ groups.

17. A compound according to claim 16 wherein R¹² is a morpholinyl, piperazinyl, thiomorpholinyl or piperidinyl group optionally substituted by one or more R¹³ or COR¹³ groups.

18. A compound according to claim 17 wherein R¹² is a morpholinyl, piperazinyl, thiomorpholinyl or piperidinyl group optionally substituted by one or more alkyl, aralkyl or CO-alkyl groups.

19. A compound according to claim 18 wherein R¹² is a morpholinyl, piperazinyl, thiomorpholinyl or piperidinyl group optionally substituted by one or more methyl, benzyl or COMe groups.

20. A compound according to claim 19 wherein R¹² is selected from the following:

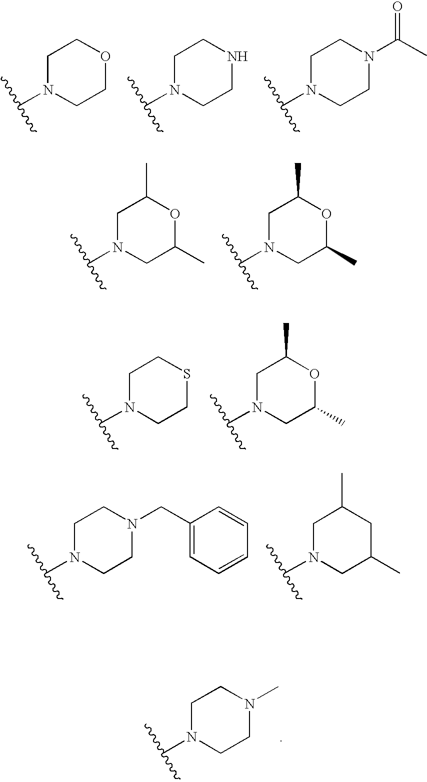

21. A compound according to claim 1 wherein each R¹² is independently selected from OH, OMe, COMe, CHO, CO₂Me, COOH, CN, CONH₂, NHMe, NH₂, NMe₂, SH, SMe, SOMe, SO₂Me, SO₂NHMe, SO₂NH₂, Cl, Br, F, I, CF₃, NO₂, N-morpholinyl, N-pyrrolidinyl, N-piperazinyl, N-thiomorpholinyl, 2,6-dimethylmorpholin-4-yl, 4-benzylpiperazin-1-yl, 3,5-dimethylpiperidin-1-yl and 4-acetylpiperazin-1-yl.

22. A compound according to claim 1 of formula Ic, or a pharmaceutically acceptable salt thereof, 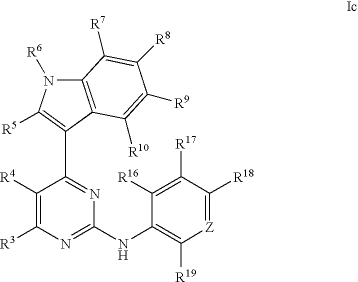
wherein
R³⁻¹⁰ are as defined in claim 1;
Z is N or CR²⁰; and
R¹⁶⁻²⁰ are each independently H, R¹¹ or R¹².

23. A compound according to claim 22 wherein Z is N.

24. A compound according to claim 22 wherein Z is CR²⁰.

25. A compound according to claim 22 wherein R¹⁶⁻²⁰ are each independently selected from H, NO₂, NR¹³R¹⁴, halogen, alkoxy and an optionally substituted heteroalicyclic group.

26. A compound according to claim 25 wherein R¹⁶⁻²⁰ are each independently selected from H, NO₂, F, OMe, N-morpholinyl, NH₂, N-pyrrolidinyl, N-piperazinyl, N-thiomorpholinyl, 2,6-dimethylmorpholin-4-yl, 4-benzylpiperazin-1-yl, 3,5-dimethyl-piperidin-1-yl and 4-acetylpiperazin-1-yl.

27. A compound according to claim 1 which is selected from the following:
4-(1H-indol-3-yl)-N-(3-nitrophenyl)pyrimidin-2-amine;
N-(4-fluorophenyl)-4-(1H-indol-3-yl)pyrimidin-2-amine;
4-(1H-indol-3-yl)-N-(6-methoxypyridin-3-yl)pyrimidin-2-amine;
4-(1H-indol-3-yl)-N-(4-morpholin-4-ylphenyl)pyrimidin-2-amine;
4-(1H-indol-3-yl)-N-(4-acetylpiperazin-1-ylphenyl)pyrimidin-2-amine;
4-(1H-indol-3-yl)-N-(4-piperazin-1-ylphenyl)pyrimidin-2-amine;
4-(1H-indol-3-yl)-N-(4-benzylpiperazin-1-ylphenyl)pyrimidin-2-amine;
4-(1H-indol-3-yl)-N-(2,6-dimethylmorpholin-4-ylphenyl)pyrimidin-2-amine;
N′-[4-(1H-indol-3-yl)pyrimidin-2-yl]-N,N-dimethylbenzene-1,4-diamine;
4-(1H-indol-3-yl)-N-(2-methyl-4-morpholin-4-ylphenyl)pyrimidin-2-amine;
4-(1H-indol-3-yl)-N-(3,4,5-trimethoxyphenyl)pyrimidin-2-amine;
4-(1H-indol-3-yl)-N-(3-methoxyl-4-morpholin-4-ylphenyl)pyrimidin-2-amine;
N-(3,5-dimethoxyphenyl)-4-(1H-indol-3-yl)pyrimidin-2-amine;
4-(1-methyl-1H-indol-3-yl)-N-(4-morpholin-4-ylphenyl)pyrimidin-2-amine;
4-(1-methyl-1H-indol-3-yl)-N-(4-acetylpiperazine-1-ylphenyl)pyrimidin-2-amine;
N-1,3-benzodioxol-5-yl-4-(1H-indol-3-yl)pyrimidin-2-amine;
4-[1-(cyclopropylcarbonyl)-1H-indol-3-yl]-N-(4-morpholin-4ylphenyl)pyrimidin-2-amine;
4-(1-acetyl-1H-indol-3-yl)-N-(4-morpholin-4ylphenyl)pyrimidin-2-amine;
4-(1H-indol-3-yl)-N-(4-methylpiperazin-1-ylphenyl)pyrimidin-2-amine;
4-(7-methoxy-1H-indol-3-yl)-N-(4-acetylpiperazin-1-ylphenyl)pyrimidin-2-amine;
4-(2-methyl-1H-indol-3-yl)-N-(4-acetylpiperazin-1-ylphenyl)]pyrimidin-2-amine;
4-(7-methyl-1H-indol-3-yl)-N-(4-acetylpiperazin-1-ylphenyl)]pyrimidin-2-amine;
4-(6-methoxy-1H-indol-3-yl)-N-(4-acetylpiperazin-1-ylphenyl)]pyrimidin-2-amine;
4-(7-chloro-1H-indol-3-yl)-N-(4-acetylpiperazin-1-ylphenyl)]pyrimidin-2-amine;
4-(6-fluoro-1H-indol-3-yl)-N-(4-acetylpiperazin-1-ylphenyl)]pyrimidin-2-amine;
4-(1H-indol-3-yl)-N-[(4-acetylpiperazin-1-yl)-3-methylphenyl]pyrimidin-2-amine;
4-(1H-indol-3-yl)-N-(3-methyl-4-thiomorpholin-4-ylphenyl)pyrimidin-2-amine;
4-(1H-indol-3-yl)-N-[(2R,6S)-2,6-dimethylmorpholin-4-ylphenyl]pyrimidin-2-amine;
4-(1H-indol-3-yl)-N-[(2S,6S)-2,6-dimethylmorpholin-4-ylphenyl]pyrimidin-2-amine;
4-(1H-indol-3-yl)-N-(3,5-dimethylpiperidin-1-ylphenyl)pyrimidin-2-amine; and
4-(1H-indol-3-yl)pyrimidin-2-amine.

28. A compound according to claim 1 which exhibits an IC₅₀ value for kinase inhibition of less than 10 μM.

29. A compound according to claim 1 which exhibits an IC₅₀ value for kinase inhibition of less than 1 μM.

30. A compound according to claim 1 which exhibits an IC₅₀ value for kinase inhibition of less than 0.1 μM.

31. A pharmaceutical composition comprising a compound according to claim 1 admixed with a pharmaceutically acceptable diluent, excipient or carrier.

32. (canceled)

33. A method of treating a proliferative disorder, said method comprising administering to a subject in need thereof, a compound of formula Ia, or a pharmaceutically acceptable salt thereof, 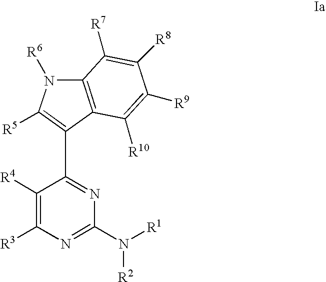
wherein R³, R⁴, R⁵, R⁶, R⁷, R⁸, R⁹, and R¹⁰ are each independently H, R¹¹ or R¹²;
R¹ and R² are each independently H, R¹¹ or R¹²; or R¹ and R² are linked to form a cyclic group together with the nitrogen to which they are attached, and wherein said cyclic group is optionally substituted with one or more R¹¹ or R¹² groups;
each R¹¹ is independently a hydrocarbyl group optionally substituted by one or more R¹² substituents;
each R¹² is independently selected from OR¹³, COR¹³, COOR¹³, CN, CONR¹³R¹⁴, NR¹³R¹⁴, SR¹³, SOR¹³, SO₂R¹³, SO₂OR¹³, SO₂NR¹³R¹⁴, R¹³, halogen, CF₃, NO₂ and an alicyclic group itself optionally substituted by one or more R¹² or R¹³ groups; and
each R¹³ and each R¹⁴ are independently H or (CH₂)ₙR¹⁵, where n is 0, 1, 2, or 3; and
each R¹⁵ is independently selected from alkyl, cycloalkyl, aryl, heteroaryl, aryl and an alicyclic group;
with the proviso that the compound is other than [4-(1H-indol-3-yl)-pyrimidin-2-yl]-[3-(1,1,2,2-tetrafluoroethoxyphenyl)]-amine;
in an amount sufficient to treat a proliferative disorder.

34. A method according to claim 33 wherein the proliferative disorder is cancer or leukemia.

35. A method according to claim 33 wherein the proliferative disorder is glomerulonephritis, rheumatoid arthritis, psoriasis or chronic obstructive pulmonary disorder.

36. A method of treating a viral disorder, said method comprising administering to a subject in need thereof, a compound of formula Ib, or a pharmaceutically acceptable salt thereof, 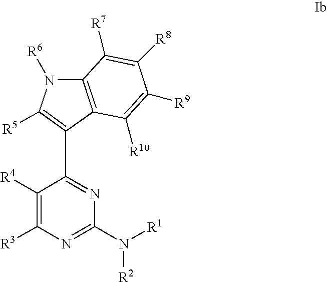
wherein R³, R⁴, R⁵, R⁶, R⁷, R⁸, R⁹, and R¹⁰ are each independently H, R¹¹ or R¹²;
R¹ and R² are each independently H, R¹¹ or R¹²; or R¹ and R² are linked to form a cyclic group together with the nitrogen to which they are attached, and wherein said cyclic group is optionally substituted with one or more R¹¹ or R¹² groups;
each R¹¹ is independently a hydrocarbyl group optionally substituted by one or more R¹² substituents;
each R¹² is independently selected from OR¹³, COR¹³, COOR¹³, CN, CONR¹³R¹⁴, NR¹³R¹⁴, SR¹³, SOR¹³, SO₂R¹³, SO₂OR¹³, SO₂NR¹³R¹⁴, R¹³, halogen, CF₃, NO₂ and an alicyclic group itself optionally substituted by one or more R¹² or R¹³ groups; and
each R¹³ and each R¹⁴ are independently H or (CH₂)ₙR¹⁵, where n is 0, 1, 2, or 3; and
each R¹⁵ is independently selected from alkyl, cycloalkyl, aryl, heteroaryl, aryl and an alicyclic group;
in an amount sufficient to treat a viral disorder.

37. A method according to claim 36 wherein the viral disorder is selected from human cytomegalovirus (HCMV), herpes simplex virus type 1 (HSV-1), human immunodeficiency virus type 1 (HIV-1), and varicella zoster virus (VZV).

38. A method of treating a CNS disorder, said method comprising administering to a subject in need thereof, a compound of formula Ib, or a pharmaceutically acceptable salt thereof, 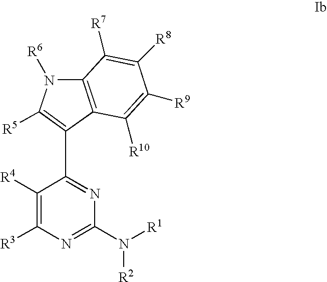
wherein R³, R⁴, R⁵, R⁶, R⁷, R⁸, R⁹, and R¹⁰ are each independently H, R¹¹ or R¹²;
R¹ and R² are each independently H, R¹¹ or R¹²; or R¹ and R² are linked to form a cyclic group together with the nitrogen to which they are attached, and wherein said cyclic group is optionally substituted with one or more R¹¹ or R¹² groups;
each R¹¹ is independently a hydrocarbyl group optionally substituted by one or more R¹² substituents;
each R¹² is independently selected from OR¹³, COR¹³, COOR¹³, CN, CONR¹³R¹⁴, NR¹³R¹⁴, SR¹³, SOR¹³, SO₂R¹³, SO₂OR¹³, SO₂NR¹³R¹⁴, R¹³, halogen, CF₃, NO₂ and an alicyclic group itself optionally substituted by one or more R¹² or R¹³ groups; and
each R¹³ and each R¹⁴ are independently H or (CH₂)ₙR¹⁵, where n is 0, 1, 2, or 3; and
each R¹⁵ is independently selected from alkyl, cycloalkyl, aryl, heteroaryl, aryl and an alicyclic group;
in an amount sufficient to treat a CNS disorder.

39. A method according to claim 38 wherein the CNS disorder is Alzheimer's disease or bipolar disorder.

40. A method of treating alopecia, said method comprising administering to a subject in need thereof, a compound of formula Ib, or a pharmaceutically acceptable salt thereof, 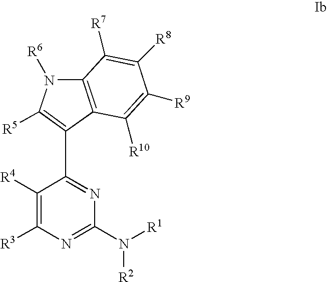
wherein R³, R⁴, R⁵, R⁶, R⁷, R⁸, R⁹, and R¹⁰ are each independently H, R¹¹ or R¹²;
R¹ and R² are each independently H, R¹¹ or R¹²; or R¹ and R² are linked to form a cyclic group together with the nitrogen to which they are attached, and wherein said cyclic group is optionally substituted with one or more R¹¹ or R¹² groups;
each R¹¹ is independently a hydrocarbyl group optionally substituted by one or more R¹² substituents;
each R¹² is independently selected from OR¹³, COR¹³, COOR¹³, CN, CONR¹³R¹⁴, NR¹³R¹⁴, SR¹³, SOR¹³, SO₂R¹³, SO₂OR¹³, SO₂NR¹³R¹⁴, R¹³ halogen, CF₃, NO₂ and an alicyclic group itself optionally substituted by one or more R¹² or R¹³ groups; and
each R¹³ and each R¹⁴ are independently H or (CH₂)ₙR¹⁵, where n is 0, 1, 2, or 3; and
each R¹⁵ is independently selected from alkyl, cycloalkyl, aryl, heteroaryl, aryl and an alicyclic group;
in an amount sufficient to treat alopecia.

41. A method of treating a stroke, said method comprising administering to a subject in need thereof, a compound of formula Ib, or a pharmaceutically acceptable salt thereof, 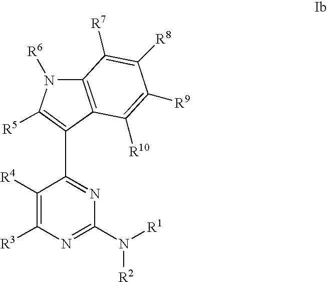
wherein R³, R⁴, R⁵, R⁶, R⁷, R⁸, R⁹ and R¹⁰ are each independently H, R¹¹ or R¹²;
R¹ and R² are each independently H, R¹¹ or R¹²; or R¹ and R² are linked to form a cyclic group together with the nitrogen to which they are attached, and wherein said cyclic group is optionally substituted with one or more R¹¹ or R¹² groups;
each R¹¹ is independently a hydrocarbyl soup optionally substituted by one or more R¹² substituents;
each R¹² is independently selected from OR¹³, COR¹³, COOR¹³, CN, CONR¹³R¹⁴, NR¹³R¹⁴, SR¹³, SOR¹³, SO₂R¹³, SO₂OR¹³, SO₂NR¹³R¹⁴, R¹³, halogen, CF₃, NO₂ and an alicyclic group itself optionally substituted by one or more R¹² or R¹³ groups; and
each R¹³ and each R¹⁴ are independently H or (CH₂)ₙR¹⁵, where n is 0, 1, 2, or 3; and
each R¹⁵ is independently selected from alkyl, cycloalkyl, aryl, heteroaryl, aryl and an alicyclic group;
in an amount sufficient to treat a stroke.

42. A method according to any one of claims 33, 36, 38, 40 or 41 wherein the compound is administered in an amount sufficient to inhibit at least one CDK enzyme.

43. A method according to claim 42 wherein the CDK enzyme is CDK1, CDK2, CDK3, CDK4, CDK6, CDK7, CDK8 and/or CDK9.

44. A method according to any one of claims 33, 36, 38, 40 or 41 wherein the compound is administered in an amount sufficient to inhibit aurora kinase.

45. A method according to any one of claims 33, 36, 38, 40 or 41 wherein the compound is administered in an amount sufficient to inhibit FLT3.

46. A method of treating diabetes or diabetic neuropathy, said method comprising administering to a subject in need thereof, a compound of formula Ib, or a pharmaceutically acceptable salt thereof, 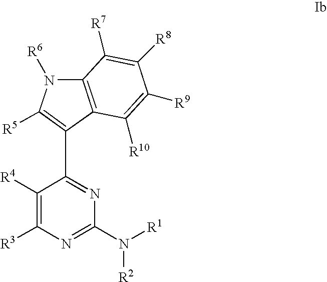
wherein R³, R⁵, R⁶, R⁷, R⁸, R⁹, and R¹⁰ are each independently H, R¹¹ or R¹²;
R¹ and R² are each independently H, R¹¹ or R¹²; or R¹ and R² are linked to form a cyclic group together with the nitrogen to which they are attached, and wherein said cyclic group is optionally substituted with one or more R¹¹ or R¹² groups;
each R¹¹ is independently a hydrocarbyl group optionally substituted by one or more R¹² substituents;
each R¹² is independently selected from OR¹³, COR¹³, COOR¹³, CN, CONR¹³R¹⁴, NR¹³R¹⁴, SR¹³, SOR¹³, SO₂R¹³, SO₂OR¹³, SO₂NR¹³R¹⁴, R¹³, halogen, CF₃, NO₂ and an alicyclic group itself optionally substituted by one or more R¹² or R¹³ groups; and
each R¹³ and each R¹⁴ are independently H or (CH₂)ₙR¹⁵, where n is 0, 1, 2, or 3; and
each R¹⁵ is independently selected from alkyl, cycloalkyl, aryl, heteroaryl, aryl and an alicyclic group;
in an amount sufficient to treat diabetes or diabetic neuropathy.

47. A method according to claim 46 wherein the diabetes is Type II diabetes.

48. A method according to claim 46 wherein the compound is administered in an amount sufficient to inhibit GSK.

49. A method according to claim 48 wherein the compound is administered in an amount sufficient to inhibit GSK3β.

50. A method of treating one or more of a microbial infection, a fungal disorder, a parasitic disorder, an inflammatory disorder, and a cardiovascular disorder, said method comprising administering to a subject in need thereof, a compound of formula Ib, or a pharmaceutically acceptable salt thereof, 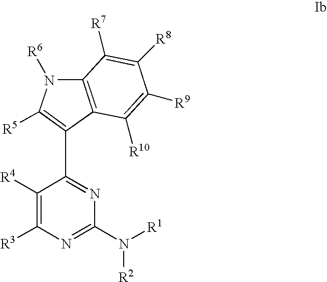
wherein R³, R⁴, R⁵, R⁶, R⁷, R⁸, R⁹, and R¹⁰ are each independently H, R¹¹ or R¹²;
R¹ and R² are each independently H, R¹¹ or R¹²; or R¹ and R² are linked to form a cyclic group together with the nitrogen to which they are attached, and wherein said cyclic group is optionally substituted with one or more R¹¹ or R¹² groups;
each R¹¹ is independently a hydrocarbyl group optionally substituted by one or more R¹² substituents;
each R¹² is independently selected from OR¹³, COR¹³, COOR¹³, CN, CONR¹³R¹⁴, NR¹³R¹⁴, SR¹³, SOR¹³, SO₂R¹³, SO₂OR¹³, SO₂NR¹³R¹⁴, R¹³, halogen, CF₃, NO₂ and an alicyclic group itself optionally substituted by one or more R¹² or R¹³ groups; and
each R¹³ and each R¹⁴ are independently H or (CH₂)ₙR¹⁵, where n is 0, 1, 2, or 3; and
each R¹⁵ is independently selected from alkyl, cycloalkyl, aryl, heteroaryl, aryl and an alicyclic group;
in an amount sufficient to treat one or more of a microbial infection, a fungal disorder, a parasitic disorder, an inflammatory disorder, and a cardiovascular disorder.

51. A method of identifying further candidate compounds capable of inhibiting one or more of a cyclin dependent kinase, an aurora kinase, FLT3, and a glycogen synthase kinase, comprising using a compound of formula Ib, or a pharmaceutically acceptable salt thereof, as defined in claim 36, in an assay for identifying further candidate compounds capable of inhibiting one or more of a cyclin dependent kinase, an aurora kinase, FLT3, and a glycogen synthase kinase.

52. A method according to claim 51 wherein said assay is a competitive binding assay.

53. A method according to any one of claims 33, 36, 38, 40, 41, 46, or 50 wherein the compound is as defined in claim 1.

54. A process for preparing a compound of formula Ib as defined in claim 36, said process comprising the steps of: 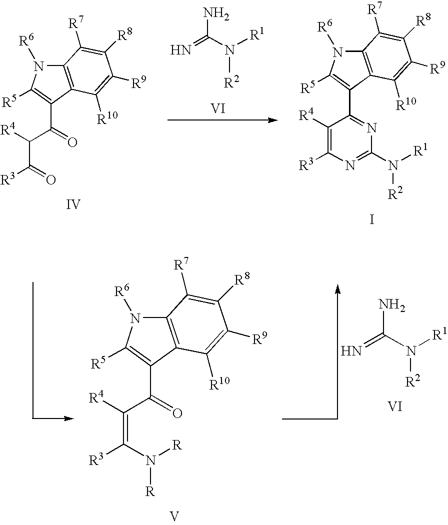
(a) condensing a compound of formula IV with a guanidine of formula VI to form a compound of formula I; or
(b) (i) converting a compound of formula IV to a compound of formula V; and
(ii) condensing said compound of formula V with a guanidine of formula VI to form a compound of formula I.

55. A process according to claim 54 wherein said compound of formula IV is prepared by acylating a compound of formula III

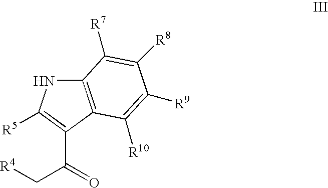

56. A process according to claim 55 wherein said compound of formula III is prepared by acylating a compound of formula II with an acid anhydride or acid halide derivative of R⁴CH₂COOH

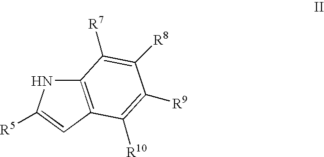

57. A process according to claim 55 wherein said compound of formula III is prepared by a process which comprises treating a compound of formula II with (i) zinc chloride and ethylmagnesium bromide, and (ii) acetyl chloride.

58. A method of treating an aurora kinase-dependent disorder, said method comprising administering to a subject in need thereof, a compound of formula 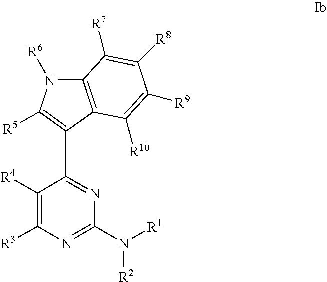
wherein R³, R⁴, R⁵, R⁶, R⁷, R⁸, R⁹, and R¹⁰ are each independently H, R¹¹ or R¹²;
R¹ and R² are each independently H, R¹¹ or R¹²; or R¹ and R² are linked to form a cyclic group together with the nitrogen to which they are attached, and wherein said cyclic group is optionally substituted with one or more R¹¹ or R¹² groups;
each R¹¹ is independently a hydrocarbyl group optionally substituted by one or more R¹² substituents;
each R¹² is independently selected from OR¹³, COR¹³, COOR¹³, CN, CONR¹³R¹⁴, NR¹³R¹⁴, SR¹³, SOR¹³, SO₂R¹³, SO₂OR¹³, SO₂NR¹³R¹⁴, R¹³, halogen, CF₃, NO₂ and an alicyclic group itself optionally substituted by one or more R¹² or R¹³ groups; and
each R¹³ and each R¹⁴ are independently H or (CH₂)ₙR¹⁵, where n is 0, 1, 2, or 3; and
each R¹⁵ is independently selected from alkyl, cycloalkyl, aryl, heteroaryl, aryl and an alicyclic group, or a pharmaceutically acceptable salt thereof, in an amount sufficient to inhibit aurora kinase.

59. A method of treating a FLT3-dependent disorder, said method comprising administering to a subject in need thereof, a compound of formula Ib 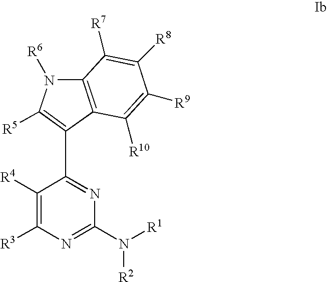
wherein R³, R⁴, R⁵, R⁶, R⁷, R⁸, R⁹, and R¹⁰ are each independently H, R¹¹ or R¹²;
R¹ and R² are each independently H, R¹¹ or R¹²; or R¹ and R² are linked to form a cyclic group together with the nitrogen to which they are attached, and wherein said cyclic group is optionally substituted with one or more R¹¹ or R¹² groups;
each R¹¹ is independently a hydrocarbyl group optionally substituted by one or more R¹² substituents;
each R¹² is independently selected from OR¹³, COR¹³, COOR¹³, CN, CONR¹³R¹⁴, NR¹³R¹⁴, SR¹³, SOR¹³, SO₂R¹³, SO₂OR¹³, SO₂NR¹³R¹⁴, R¹³, halogen, CF₃, NO₂ and an alicyclic group itself optionally substituted by one or more R¹² or R¹³ groups; and
each R¹³ and each R¹⁴ are independently H or (CH₂)ₙR¹⁵, where n is 0, 1, 2, or 3; and
each R¹⁵ is independently selected from alkyl, cycloalkyl, aryl, heteroaryl, aryl and an alicyclic group, or a pharmaceutically acceptable salt thereof, in an amount sufficient to inhibit FLT3.

60. A method of treating an CDK-dependent disorder, said method comprising administering to a subject in need thereof, a compound of formula Ib 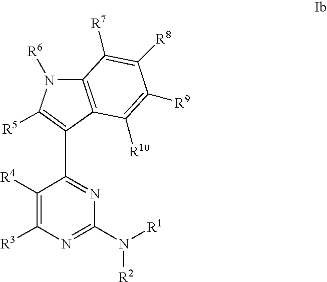
wherein R³, R⁴, R⁵, R⁶, R⁷, R⁸, R⁹, and R¹⁰ are each independently H, R¹¹ or R¹²;
R¹ and R² are each independently H, R¹¹ or R¹²; or R¹ and R² are linked to form a cyclic group together with the nitrogen to which they are attached, and wherein said cyclic group is optionally substituted with one or more R¹¹ or R¹² groups;
each R¹¹ is independently a hydrocarbyl group optionally substituted by one or more R¹² substituents;
each R¹² is independently selected from OR¹³, COR¹³, COOR¹³, CN, CONR¹³R¹⁴, NR¹³R¹⁴, SR¹³, SOR¹³, SO₂R¹³, SO₂OR¹³, SO₂NR¹³R¹⁴, R¹³, halogen, CF₃, NO₂ and an alicyclic group itself optionally substituted by one or more R¹² or R¹³ groups; and
each R¹³ and each R¹⁴ are independently H or (CH₂)ₙR¹⁵, where n is 0, 1, 2, or 3; and
each R¹⁵ is independently selected from alkyl, cycloalkyl, aryl, heteroaryl, aryl and an alicyclic group, or a pharmaceutically acceptable salt thereof, in an amount sufficient to inhibit a cyclin dependent kinase.

61. A method of treating a GSK-dependent disorder, said method comprising administering to a subject in need thereof, a compound of formula Ib 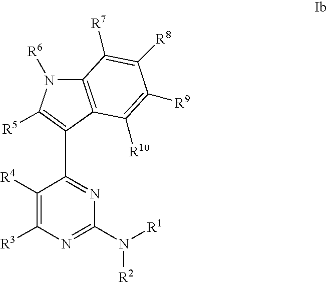
wherein R³, R⁴, R⁵, R⁶, R⁷, R⁸, R⁹, and R¹⁰ are each independently H, R¹¹ or R¹²;
R¹ and R² are each independently H, R¹¹ or R¹²; or R¹ and R² are linked to form a cyclic group together with the nitrogen to which they are attached, and wherein said cyclic group is optionally substituted with one or more R¹¹ or R¹² groups;
each R¹¹ is independently a hydrocarbyl group optionally substituted by one or more R¹² substituents;
each R¹² is independently selected from OR¹³, COR¹³, COOR¹³, CN, CONR¹³R¹⁴, NR¹³R¹⁴, SR¹³, SOR¹³, SO₂R¹³, SO₂OR¹³, SO₂NR¹³R¹⁴, R¹³, halogen, CF₃, NO₂ and an alicyclic group itself optionally substituted by one or more R¹² or R¹³ groups; and
each R¹³ and each R¹⁴ are independently H or (CH₂)ₙR¹⁵, where n is 0, 1, 2, or 3; and
each R¹⁵ is independently selected from alkyl, cycloalkyl, aryl, heteroaryl, aryl and an alicyclic group, or a pharmaceutically acceptable salt thereof, in an amount sufficient to inhibit GSK.
